(19) United States
(12) Patent Application Publication
(10) Pub. No.: US 2008/0312523 A1
(43) Pub. Date: Dec. 18, 2008

(54) Apparatus and Method for Reducing Interference

(76) Inventor: William James Ross Dunseath, Silver City, NM (US)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/629,237

(22) PCT Filed: Jun. 7, 2005
(86) PCT No.: PCT/EP05/06126

§ 371 Date: Jul. 8, 2008

(30) Foreign Application Priority Data
Mar. 3, 2005 (EP) 05251283.7

(51) Int. Cl. A61B 5/055; A61B 5/053

(52) U.S. Cl. 600/383; 600/411

(57) ABSTRACT

An electronic apparatus for reducing interference in a desired signal, the apparatus comprising: (a) a plurality of measurement signal lines, each connected to a respective measurement signal electrode; and (b) one or more reference signal lines, each connected to respective one or more reference electrodes; each of said measurement signal lines or a respective group of said measurement signal lines being associated by being in close physical proximity with a respective one of said reference signal lines for a substantial part of their lengths, so that each measurement signal line or signal line group with its corresponding reference signal line forms a measurement signal line or measurement signal line group/reference signal line pair, said electronic apparatus further comprising subtraction means for subtracting an interference on each reference signal line from an interference signal on the associated measurement signal line or from each measurement signal line in the measurement signal line group in that measurement signal line or measurement signal line group/reference signal line pair, wherein at least one of the measurement signal electrodes is arranged to be in direct electrical connection with a subject and at least one of the reference signal electrodes is arranged to be in close physical proximity but not in direct electrical contact with the subject.

### FIELD OF THE INVENTION

This present invention relates to an electronic method and apparatus for reducing interference in a signal wherein the interference is of a large magnitude relative to the data component to be extracted from the signal. It is particularly, although not exclusively, suited to reducing noise in biopotential signal acquisition, which noise is caused by electrical and magnetic fields. It may also be used in other applications such as semiconductor physics, where electrical signals may be derived under conditions where a large noise component is present, e.g. due to a large varying magnetic field.

### BACKGROUND OF THE INVENTION

Functional magnetic resonance imaging (fMRI) is widely used in both medical and non-medical imaging to obtain a spatial image of “slices” through the brain. In the medical context, MRI is used to identify lesions such as areas of restricted blood flow or tumours. Outside the medical field, fMRI has, for example, been a useful tool in cognitive neuroscience for investigating brain response to various external stimuli.

Electroencephalography (EEG) has traditionally been used for investigations into brain activity. It may, for example, be used to investigate abnormal brain activity in disease states such as epilepsy or in certain psychiatric abnormalities.

If fMRI and EEG could be used together, they could advantageously combine both spatial and temporal information about brain function which would be of major benefit for both medical and non-medical uses. However, an EEG signal obtained from a scalp electrode is in the range typically of 10 μV to 100 μV at an impedance of around 500Ω to 50K Ω. The large magnetic and radio frequency (rf) fields produced by MRI machines swamp this signal with induced noise on the signal wire. In particular, switching of the MRI magnetic gradients causes extraneous pulses in the EEG signal.

However, at least two other sources of interference tend to occur in such a system. The first is powerline (mains) interference from the AC power system (typically 50 Hz or 60 Hz). The second is ballistocardiogram (BCG) noise, ie noise caused by the pulsing blood flow of the subject interacting with the large static magnetic field of the MRI scanner.

Conventional known methods for rejecting interference in EEG include the use of a reference electrode and differential amplifier, electrical isolation of the EEG amplifiers, shielding of the electrode lead wires, driving the shield of the lead wires with a common mode voltage, and electrical filtering of the EEG signal. Additional strategies have been employed for EEG in fMRI, such as the use of carbon lead wires and inductors.

For example, U.S. Pat. No. 5,445,162 proposes a system using electrodes and wiring designed to minimise noise pick-up and the fMRI and EEG data are obtained alternately. It proposes locating the EEG recording equipment outside the MRI room to minimise interference.

U.S. Pat. No. 5,513,649 proposes a system for removing contaminants from EEG recordings. It proposes that an adaptive filter is used to estimate the contaminants in the measured EEG data and then subtracts them from the primary signal to obtain the corrected EEG data.

WO-A-03/073929 discusses the potential problems associated with concurrent fMRI and EEG measurements, namely noise induced in the EEG signal by the rf and magnetic fields (as mentioned above) and the disruption to the fMRI measurement by introduction of ferromagnetic material in the EEG electrodes, into the bore of the fMRI machine. This reference comments upon possibilities for alleviating these problems. One is to dispense with ferromagnetic materials in the EEG electrodes and to use an alternative such as carbon fibre. Another is to rearrange the EEG leads to minimise interference with the rf field.

The aforementioned WO-A-03/073929 also recognises safety problems inherent in deploying EEG equipment inside a pulsed rf field, eg due to induced currents. Solutions to these problems have included raising the impedance of the EEG detection circuit by means of resistors or by using different electrode systems or different electrode materials, or by incorporating a fibre optic link in the line between the electrodes and the circuit. The reference proposes that a better method of avoiding such hazards is to incorporate an amplifier within the electrode structure.

WO-A-02/13689 describes a method of reducing interference in EEG, ECG and EMG, especially in combination with MRI whereby pairs of electrodes are connected to differential amplifiers. An interference signal is obtained by synchronisation of measurement signals with a timing signal which initiates digitisation of the signals. Subtraction of the interference is then effected digitally.

Despite these numerous proposals, there still remains a need for a system whereby truly simultaneous derivation of EEG and fMRI signals could be made possible, by eliminating the several major sources of interference on the EEG signal at an early stage in the processing circuitry rather than removing it by post-processing.

In principle, any one of a number of electrophysiological measurement systems can be combined with fMRI, instead of or in addition to EEG. Examples of these are electrocardiography (ECG), electromyography (EMG), electro-oculography (EOG), electroretinography (ERG) and galvanic skin response measurement (GSR). The same problems can occur with any electrophysiological measurement such as these, when used in combination with MRI, for example fMRI. Therefore, there is a need to suppress interference sufficiently when simultaneously conducting any electrophysiological measurement in combination with fMRI. For convenience, for the generic term electrophysiological measurement, hereinafter the abbreviation EPM will be used. The present invention is useful with any of these, or other EPM systems. It is also useful in other combinations of an EPM with interventions which utilise a large magnetic field, for example, transcranial magnetic stimulation (TMS).

### SUMMARY OF THE INVENTION

A first aspect of the present invention provides an electronic apparatus for reducing interference in a desired signal, the apparatus comprising:—
(a) a plurality of measurement signal lines, each connected to a respective measurement signal electrode; and
(b) one or more reference signal lines, each connected to a respective one or more reference electrodes;
   each of said measurement signal lines or a respective group of said measurement signal lines being associated by being in close physical proximity with a respective one of said reference signal lines for a substantial part of their lengths, so that each measurement signal line or signal line group with its corresponding reference signal line forms a measurement signal line or measurement signal line group/reference signal line pair, said electronic apparatus further comprising subtraction means for subtracting an interference signal on each reference signal line from an interference signal on the associated measurement signal line or from each measurement signal line in the measurement signal line group in that measurement signal line or measurement signal line group/reference signal line pair;
   wherein at least one of the measurement signal electrodes is arranged to be in direct electrical connection with a subject and at least one of the reference signal electrodes is arranged to be in close physical proximity but not in direct electrical contact with the subject.

A second aspect of the present invention provides a method of reducing interference from a desired signal, the method comprising
(a) providing a plurality of measurement signal lines, each carrying a desired signal and an interference signal;
(b) providing one or more reference signal lines, each carrying at least an interference signal, each measurement signal line or a respective group of measurement signal lines being associated by being in close physical proximity for a substantial part of its length with a respective reference signal line to provide respective measurement signal line or measurement signal line group/reference signal line pairs; and
(c) performing a subtraction step of subtracting the interference signal on each respective reference signal line from the interference signal on the associated measurement signal line or from each measurement signal line in the measurement signal line group of its measurement signal line or measurement signal line group/reference line pair;
   wherein at least one of the measurement signal electrodes is arranged to be in direct electrical connection with a subject and at least one of the reference signal electrodes is arranged to be in close physical proximity but not in direct electrical contact with the subject.

As used herein and unless specifically stated to the contrary, the unqualified term “signal line” means a measurement signal line deriving a primary measurement signal, as opposed to a reference (signal) line or ground line.

Each measurement signal line may be associated with its own reference signal line or the measurement signal lines may be grouped onto one or more groups each comprising a plurality of measurement signal lines each having its own at least one associated reference signal line. A combination of these arrangements is also possible.

As used herein, “direct electrical contact” preferably means a contact resistance of 10K ohms or less, preferably 1K ohms or less and “not in direct electrical contact” is to be construed accordingly. In some preferred embodiments, “direct electrical contact” as used herein preferably means a contact resistance of 1K ohms or less, preferably 100 ohms or less and “not in direct electrical contact” is to be construed accordingly.

As used herein, the term “group” preferably means two or more.

As will be explained in more detail hereinbelow, the reference signal electrode(s) are preferably arranged to be reference nodes in a reference mesh which is substantially insulated from the subject.

Preferably a compensation signal line and most preferably, also an associated reference line are also provided. As a generality, a compensation signal on the compensation signal line, derived from a separate compensation line electrode, is used to reduce interference in the or each measurement signal. Preferably, the signal on the compensation signal line is processed in a compensation signal processing unit to produce a plurality of compensation signal components. The compensation signal components are respectively used to reduce interference in respective interference reduction modules which process the respective measurement signal or signals preferably after subtraction of all or part of the corresponding reference signal or signals.

A compensation signal is preferably derived from a separate compensation signal electrode connected to a neutral (relatively non-responsive) part of the subject.

Thus, in one class of embodiments, the or each measurement signal is derived via a respective measurement signal line connected to its own measurement signal electrode and for each such measurement signal line, there is a corresponding reference signal line in close proximity therewith for a substantial part of their mutual lengths (or one or more group(s) of measurement signal lines may share a single reference signal line in close proximity in the same way). Each such reference signal line is connected to a respective reference signal electrode or connection point which in use, is positioned close to its corresponding measurement signal electrode. Preferably, the compensation signal line (when utilised) is also provided with a corresponding reference signal line connected to a reference signal electrode or connection point, situated close to the compensation signal electrode. Preferably, each reference signal is at least partially subtracted from the corresponding measurement signal, or signals in the case of a shared reference signal line, (or the compensation signal, as the case may be), for example with the respective primary signal unit (or compensation signal unit). Preferably, the compensation signal line has its own reference line in close physical proximity therewith along a substantial part of their mutual lengths.

For at least some measurement signal lines and/or the compensation signal line, more than one additional reference line may be provided, connected to the same reference electrode or its own respective reference electrode. As stated above, it is also possible for one or more groups of measurement signal lines to share one or more associated reference signal lines.

Preferably also, corresponding ground connections/ground lines are provided for each signal, compensation, and reference connections or electrodes and lines, or each signal line/reference line pair and the compensation line/reference line pair shares a respective single common ground line. A ground line may also be provided for the compensation signal line and any accompanying reference line. In a particularly preferred embodiment, substantially all such ground lines are connected to a shared single ground electrode.

The interference reduction may optionally employ adaptive noise cancellation, preferably in real time, in which the amount of interference to be removed may be determined dynamically and varied over time.

Preferably, the interference reduction modules in each primary signal processing unit are arranged in series. Preferably, in each primary signal processing unit, separate interference reduction modules are provided for reducing at least two of magnetic switching interference, mains power interference, eyeblink artifact interference and ballistocardiogram interference.

In an EEG measurement employing an embodiment of the present invention, any electrodes to the human or animal skin (eg scalp) may be dry or “wet” (i.e. employing an electrically conductive gel or paste).

Any circuit element or method step independently may be implemented by analog or digital means.

The present invention may also be defined by any of the following further aspects of the invention as set-out below. Each of these may optionally also employ any essential, preferred or optional feature of any other such aspects of the invention (method or apparatus as appropriate), and/or any other essential, preferred or optional feature of any other aspect of the invention described, defined or claimed elsewhere in this specification, including in terms of any measurements, types of applications and/or use of specific electrode arrangements or electrode support apparatus.

According to a third aspect of the present invention there is provided a method of reducing interference from a desired signal, the method comprising
(a) providing a signal line carrying a desired signal and an interference signal;
(b) providing a reference line carrying at least an interference signal, said signal line and reference line being associated by being in close physical proximity for a substantial part of their lengths; and
(c) a subtraction step of subtracting the Interference signal on the reference line from the interference signal on the signal line.

Preferably, the method further comprises:
(a) deriving a compensation signal; and
(b) generating a plurality of compensation signal components from said compensation signal;
   wherein the subtraction step comprises separately subtracting at least part of each of said compensation signal components from said measurement signal.

According to a fourth aspect of the present invention there is provided an electronic apparatus for reducing interference in a desired signal, the apparatus comprising
(a) a signal line connected to a signal electrode; and
(b) a reference line connected to a reference electrode;
   said signal line and reference line being associated by being in close physical proximity for a substantial part of their lengths, said electronic apparatus further comprising subtraction means for subtracting an interference signal on the reference line from an interference signal on the signal line thereby to enhance a desired signal on the signal line.

According to a fifth aspect of the present invention there is provided an electronic apparatus for reducing interference in a signal derived from an EPM the apparatus comprising
(a) a signal line connected to a signal electrode;
(b) a reference line connected to a reference electrode; and
(c) at least one ground line for said signal line and reference line, said ground line or lines being connected to at least one ground electrode or individually to respective ground electrodes;
   said electronic apparatus further comprising subtraction means for subtracting an interference signal on the reference line from a signal on the signal line.

According to a sixth aspect of the present invention there is provided an electronic apparatus for reducing interference in a desired signal, the apparatus comprising:—
(a) a plurality of signal lines, each connected to a respective signal electrode; and
(b) one or more reference lines connected to one or more reference electrodes; and;
(c) one or more ground lines connected to one or more ground electrodes;
   said electronic apparatus further comprising subtraction means for subtracting an interference signal on the or each reference line from an interference signal on the signal lines and/or subtracting an interference signal on the or each ground line from the interference signal on the signal lines.

According to a seventh aspect of the present invention there is provided a method of reducing interference on a signal derived from an EPM, the method comprising
(a) providing a signal line carrying a desired signal and a first interference signal, said signal line being connected to a signal electrode;
(b) providing a reference line carrying at least a second interference signal, said reference line being connected to a reference electrode;
(c) providing a ground line for said signal line and reference line, said ground line or lines being connected to at least one ground electrode or individually to respective ground electrodes; and
(d) a subtraction step of subtracting the second interference signal on the reference line from the first interference signal on the signal line.

According to an eighth aspect of the present invention there is provided a method of reducing interference from a desired signal, the method comprising
(a) providing a plurality of signal lines, each carrying a desired signal and a first interference signal;
(b) providing one or more reference lines carrying at least a second interference signal;
(c) providing one or more ground lines; and
(d) performing a subtraction step of subtracting the second interference signal from said first interference signal.

At least one compensation signal line may be provided for connection to a compensation signal electrode. The compensation signal electrode is preferably located on a subject in a “neutral” position (eg in the case of EEG, on or near an ear). The resultant at least one compensation signal, delivered via the compensation signal line(s) may be used to at least partially reduce interference on the (measurement) signal line or lines, eg by a subtractive process. The compensation signal line is preferably associated with its own reference line which is preferably in close physical proximity thereto along a substantial part of their mutual lengths and is connected to a reference electrode (node) associated with the compensation signal electrode.

According to a ninth aspect of the present invention there is provided an electronic apparatus for reducing interference in a desired signal, the apparatus comprising:—
(a) a plurality of measurement signal lines, each connected to a respective measurement signal electrode; and
(b) one or more reference signal lines, each connected to a respective one or more reference electrodes;
   each of said measurement signal lines being associated by being in close physical proximity with a respective one or more of said reference signal lines for a substantial part of their lengths, so that each measurement signal line with its corresponding reference signal line forms a measurement signal line/reference signal line pair, said electronic apparatus further comprising subtraction means for subtracting an interference signal on each reference signal line or lines from an interference signal on the associated measurement signal line in that measurement signal line/reference signal line pair;
   wherein at least one of the measurement signal electrodes is arranged to be in direct electrical connection with a subject and at least one of the reference signal electrodes is arranged to be in close physical proximity but not in direct electrical contact with the subject.

This embodiment may find particular use in electrophysiological measurement systems such as ballistocardiograms (BCG) which may be combined with MRI such as fMRI.

According to a tenth aspect of the present invention there is provided a cap for supporting one or more electrodes for use in an electronic apparatus for reducing interference in a desired signal, the cap comprising:—
(a) a conductive layer; and
(b) at least one measurement signal electrode positioned for contact with a subject; at least one of the at least one measurement signal electrode or electrodes having associated therewith a reference electrode in electrical contact with the conductive layer but arranged so as not to be in use in direct electrical contact with the subject.

Preferably, the conductive layer comprises a conductive mesh.

In a preferred embodiment the cap comprises an electrode support structure apparatus for effecting an EPM, the cap further comprising:
an array of measurement signal electrodes presented for contacting the skin of a subject, first connection means being provided for independent electrical connection to each of said measurement signal electrodes, and
second connection means for independent electrical connection to the or each of said reference electrodes.

Preferably, an insulating layer is provided for insulating in use the conductive layer from the subject.

Preferably, the number of said reference electrodes is substantially the same as the number of said measurement signal electrodes.

In a preferred embodiment, each measurement signal electrode or group of signal electrodes has a corresponding respective reference electrode in close physical proximity thereto.

Preferably, said cap further supports one or more ground electrodes presented for contacting the skin of the subject in use, the cap further comprising third connection means for independent electrical connection to each of said ground electrode or electrodes.

In a preferred embodiment, the cap supports a single ground electrode and, preferably, the cap supports a compensation signal electrode.

A respective reference electrode with its own independent electrical connection is preferably provided for the ground electrode and the compensation signal electrode.

The conductive layer preferably comprises a continuous laminar-member comprising one or more of said reference electrode or electrodes.

In a preferred embodiment, said conductive layer comprises a matrix of discrete members respectively comprising one or more of said reference electrode or electrodes.

In a preferred embodiment the cap is a flexible cap.

In an alternative preferred embodiment, the cap is a rigid cap, the conductive layer being flexible.

In accordance with all aspects of the present invention, a “reference loop” is used for subtracting at least some interference signals induced by external fields into a circuit loop. In preferred embodiments described hereinbelow, this circuit loop is formed by the connection between the living body and electronic amplification circuitry. In the described embodiments, a simplified version of the reference loop is described for use in multi-channel EPM recordings, such as EEG recordings in order to reduce noise voltages induced by the magnetic fields generated in a functional magnetic resonance imaging device (fMRI). In addition, an embodiment of a complete circuit means is described for acquiring simultaneous EPM in the MRI or fMRI environment, with minimal interference to the EPM and fMRI. EPM signals such as EEG signals can still have large interference components if used also without FMRI or the like, eg generated by electric motors in the vicinity. The present invention is also useful in such applications, reducing or removing the need for screening of the noise source and/or data acquisition circuitry.

In order to achieve EPM data acquisition, concurrent with fMRI, the EPM data acquisition circuitry must reject interference caused by external (to the body) electric and magnetic fields. The main sources of interference are low frequency electric and magnetic fields from the AC power mains (commonly 50 or 60 Hz), switched magnetic fields from fMRI with fundamental frequencies ranging down to approximately 500 Hz, and radio frequency (rf) electromagnetic fields from fMRI ranging from 60 to 130 MHz. Another source of interference is ballistocardiogram noise due to pulsing of circulatory blood in the magnetic field. In addition, the large static magnetic field of the MRI scanner causes interference voltage to be induced in EPM signal lines whenever movement of the electrodes or lead wires occurs. At least two of these are reduced as separate interference components in accordance with the first and second aspects of the present invention.

A single signal line can be connected to a respective separate signal electrode. A reference line may be connected to a single reference electrode or to a respective separate reference electrode or any other arrangement involving multiple reference electrodes.

Each signal line (or group of signal lines) may therefore be associated with a corresponding one of the reference lines to be in close proximity for a substantial part of their lengths, so that each respective signal line and associated reference line constitutes a respective signal line (or signal line group)/reference line pair. The subtraction means is then arranged to subtract an interference signal on each reference line from the interference signal on its associated signal line (or each signal line of the respective group) in the pair, thereby enhancing the desired signal on that signal line.

In preferred embodiments of the invention, at least one reference line is connected to a conductive member physically close to, but not in direct electrical contact with part of the human or animal body (eg the scalp in the case of an EEG measurement). This conductive member may, for example, be in the form of a conductive mesh.

Essential for some, whilst merely preferable for other aspects of the present invention is provision of one or more ground lines. Any signal line/reference line pair may share a common ground line, preferably in close physical proximity with both, or each signal line and reference line may be provided with its own ground line, preferably in close physical proximity therewith. A combination of such arrangements is also possible (one or more shared ground lines for some signal/reference line pairs and one or more individual ground lines for any one or more others). All ground lines may be connected to a common ground electrode or to individual respective ground electrodes, or any other arrangements involving multiple ground electrodes. Preferably, the or each ground electrode is in direct (low resistance) contact with the subject (eg the skin of the head or scalp in the case of EEG), as described further hereinbelow. In an especially preferred class of embodiments, a plurality of measurement signal lines has each connected to a respective measurement signal electrode. Each measurement signal line (or group of measurement signal lines) has its own associated reference signal line connected to a respective reference signal electrode (node). A separate ground electrode is connected to a ground line and a separate compensation signal electrode is connected to a compensation signal line. The compensation signal line and ground line each have a respective associated reference line connected to a dedicated additional respective reference electrode.

Where an individual line or lines (measurement signal, compensation signal, reference signal or ground) is or are connected to its, or their, own dedicated electrode (signal, reference, or ground, respectively), that electrode may be embodied as two or more electrode entities with the reference line or lines being connected thereto in parallel. The terms “electrode” and “node” (see below) are to be interpreted as encompassing these possibilities, except where explicitly stated to the contrary or where the context forbids.

The or each measurement signal line, compensation signal line and/or ground line, as the case may be, may be in close physical proximity for a substantial part of the length thereof, with a respective reference line, a respective ground line, or both, preferably twisted together therewith.

Preferably, signal and any ground electrodes are in direct electrical connection with the subject (usually the head, or head/neck region when the EPM is EEG, e.g. mainly to the scalp). This preferably means an individual electrode contact resistance of less than 1 Kohms. However, reference electrodes are preferably not in direct electrical contact with the subject but are electrodes in close physical proximity with the subject, preferably each respectively close to its associated signal electrode.

Preferably, and particularly when the EPM is EEG the reference electrodes are arranged as a mesh. Then signal and reference electrodes may be arranged over the head or scalp but one signal/reference electrode pair may be attached to positions where the pick-up of physiological electrical signals will be low, such as beneath the ear. However, at least one reference electrode is electrically isolated from the subject. Thus, it is to be understood that the term “electrode” includes variants which are not in direct electrical contact with the subject.

A preferred form of construction comprises a flexible, electrically conductive elastic reference mesh material acting as a cap to hold the electrodes in place. The reference mesh material may be coated with an insulating layer to electrically isolate the mesh from the body and electrodes. All components are preferably made from materials chosen to be resistant to chemical disinfectants and detergents.

In a preferred embodiment the apparatus further comprises an electrode support structure apparatus for effecting an EPM, the apparatus comprising an electrode support having supported thereon, an array of measurement signal electrodes presented for contacting the skin of a subject, first connection means being provided for independent electrical connection to each of said measurement signal electrodes, the apparatus further comprising an electrically conductive mesh having one or more of reference nodes and second connection means for independent electrical connection to the or each of said reference nodes. This support structure may be employed with any circuit, method or apparatus according to any other aspect of the present invention.

As used herein, any electrical contact point to a reference mesh is usually termed an “electrode”. However, the term “node” is also used for such a contact point with a reference mesh and as such, can be considered synonymous with electrode, whether or not any part of the mesh is in direct electrical contact with the subject, eg with the skin of the subject.

One suitable form of construction is in the form of a rigid or flexible cap, preferably having two layers of insulating elastic cap material with an electrically conductive reference mesh construction (preferably flexible) sandwiched between, and electrodes anchored to the cap. Cap structures for supporting EEG electrodes are already known from WO-A-00/27279 and U.S. Pat. No. 6,708,051.

Each electrode site on any suitable cap structure, may for example have four wires—two for the signal loop and two for the reference loop—arriving as two twisted pairs twisted around each other. One wire connects to the body electrode; one wire connects to the reference mesh next to the electrode; one wire proceeds across the cap to the body ground electrode; and one wire proceeds across the cap to the reference mesh ground connection. A multichannel arrangement would comprise a plurality (n) of such sites.

Reference mesh material can be made of carbon loaded fabrics, foam or yarns (carbon wire). Other conductive materials can be used for loading in addition to or in lieu of carbon, such as a silver-coated polymer substrate, eg nylon.

For the avoidance of doubt, reference to subtraction in accordance with any aspect of the present invention means any attenuation of interference on a signal line by deriving an interference signal from a corresponding reference line and using it to diminish the interference signal on the signal line. Arithmetic subtraction as well as other operations are included within this term. The definition includes substantial total elimination of the interference signal but also covers at least some diminution of the interference signal from the signal line.

Reference herein to any two or more lines being associated in close proximity for a substantial part of their length(s) preferably means that the respective lines run in close physical proximity for at least 50%, more preferably at least 60%, still more preferably at least 70%, yet more preferably still at least 80% and most preferably at least 90% of their lengths (when one or more wires is longer than any other relevant wire, then these percentages are of the longest).

Any lines which are in close proximity may be arranged thus by any suitable means, eg coaxially (such as with the reference line surrounding a core of the signal line, or vice versa) or by being run together as a twin wire pair (or multi-wire group) or by any other means, but most preferably, by being twisted together.

The subtraction means preferably comprises a differential amplifier with inverting and non-inverting inputs connected to signal line(s) and reference line(s) respectively.

Each signal line/reference line pair may be shielded, for example by a metallic sheathing which suitably may be connected to a ground connection.

The subtraction means may also comprise one or more common mode chokes associated with the respective signal line/reference line pairs, the windings of each such common mode choke being connected to a respective one of the signal line and the reference line. The subtraction means preferably also comprises low pass filter means, especially a seventh order low pass filter, an exemplary embodiment of which comprises a 0.05° Equiripple-type filter.

The apparatus and method of any aspect of the present invention may be deployed in the MRI room itself, although recording may be conducted outside that room. The apparatus of any aspect of the present invention may be substantially totally electrically wired, ie not require any optical or wireless link, although the latter are also possible.

One or more preferred embodiments of the present invention provide for substantially simultaneous data acquisition and read-out, thus providing minimal lag between data acquisition and data availability, as may otherwise arise due to post-processing, for example.

The electronic circuit and interference reduction method of one or more preferred embodiments of the present invention may be employed with any measurement signal subject to interference but especially for any EPM alone or in combination with MRI, FMRI or TMS. It can also be used to reduce interference on signals obtained from magnetoencephalography (MEG). MEG is a technique analogous to EEG which instead of using an electrode on the surface of the head, uses an array of sensors to measure change in magnetic fields outside the skull generated by neuronal activity.

As will be explained further hereinbelow, the present invention is also useful in the application of medical or quasi-medical measurements, other than EEG.

The present invention will now be explained in more detail by way of the following description of preferred embodiments, and with reference to the accompanying drawings, in which:—

### DESCRIPTION OF THE DRAWINGS

FIG. 1 shows a schematic of an EEG and fMRI set up, in which an interference reduction apparatus according to an embodiment of the present invention may be employed;

FIG. 2 shows the fMRI pulse sequence employed in the set-up of FIG. 1;

FIG. 3 shows a circuit diagram of an example of an electronic interference reduction apparatus;

FIG. 4 shows a block schematic diagram of a further example of an electronic interference reduction apparatus,

FIG. 5 shows a circuit diagram of the system of FIG. 4;

FIG. 6 shows an equivalent circuit for the reference loop arrangement of a single channel for use in the circuits of FIGS. 3 to 5 utilising a reference electrode, and a ground electrode connected to a body;

FIG. 7 shows an equivalent circuit for demonstrating another source of interference;

FIG. 8 shows an equivalent circuit for a section of multiple signal electrodes S1 to Sn mounted on a body with an accompanying reference loop network or mesh;

FIG. 9 shows suitable amplification, subtraction and filtering circuitry for use with arrangements generally as depicted in FIG. 8;

FIG. 10 shows front-end circuitry forming part of a particularly preferred embodiment of the present invention which utilises reference electrodes and ground electrodes;

FIG. 11 shows side views of EEG electrode connections to a human head for use in an embodiment which comprises the circuitry shown in FIG. 10;

FIG. 12 shows side views of reference mesh connections for use in an embodiment which comprises the circuitry shown in FIG. 10;

FIG. 13 shows the arrangement of scan head and circuitry for the embodiment of FIGS. 10-12, with respect to the shielded scanner room;

FIGS. 14 and 15 show intermediate circuitry inside a shielded amplifier enclosure, which receives signals from the front-end circuitry shown in FIG. 10;

FIG. 16 shows the location of the circuitry of FIGS. 14 and 15 within the shielded amplifier enclosure, relative to the shielded scanner room and exterior control room;

FIG. 17 shows a front end circuit diagram of an alternative embodiment of a noise reduction circuit according to the present invention;

FIG. 18 shows the circuitry of filters downstream of the front end shown in FIG. 17;

FIG. 19 shows a front end circuit diagram of an embodiment of the present invention, employing electrical isolation of reference loop ground lines;

FIG. 20 shows a perspective view of an electrode cap according to, and for use in, the present invention; and

FIG. 21 shows a cross section through one electrode region of the electrode cap shown in FIG. 20.

### DESCRIPTION OF PREFERRED EMBODIMENTS

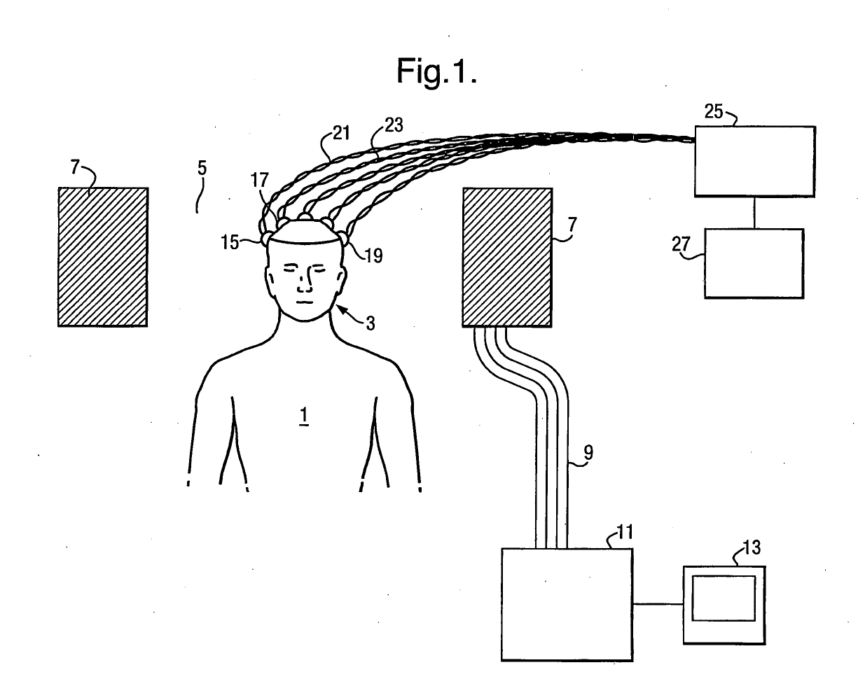

FIG. 1 shows a basic fMRI and EEG system in which the apparatus and method of one or more embodiments of the present invention may be employed.

As shown in FIG. 1, a subject 1 is arranged with the subject's head 3 located within the bore 5 of an fMRI coil unit 7 which carries magnetic field windings and rf coils. These coils and windings are energised via a multiplicity of wiring connections 9 etc which connect the coil unit 7 to operational circuitry 11. The operational circuitry unit is connected to a memory and display unit 13 whereby the MRI scans can be stored, displayed and printed at will.

A plurality of electrodes 15, 17, 19 etc for obtaining EEG signals are attached to the scalp of the subject 1. As will be explained in more detail hereinbelow, one of these electrodes 19 is a “reference electrode”. Signals from the electrodes 15, 17, 19 etc are conveyed by wires 21, 23 etc to an EEG control unit 25 which is connected to a recorder 27 situated outside the MRI room.

The combined fMRI/EEG arrangement may be considered to apply to any specific embodiment of EEG processing circuitry described hereinbelow.

In a worked embodiment, the MRI system used for obtaining data presented in more detail hereinbelow was the Siemens Allegra™ (3.0T)-MR6.

The Siemens Allegra™ 3T is a head-only research magnet. It has the necessary hardware and software to perform basic and clinical scans. Gradient hardware consists of a 36 cm I.D. asymmetric gradient coil capable of imaging at 60 mT/m with slew rates in excess of 600 Tim/s at a duty cycle of 70% allowing single shot echoplanar imaging (EPI) at a sustained rate of 14 images/second. The system has a 15 kW RF amplifier, and 8 RF preamp channels for this system supports the Syngo™ software on a Windows™ NT platform.

The EPI regime employed 1 to 13 gradient switching pulses (images) per second. Gradient strength: 20-35 mT/m, max 40 mT/m; Slew rate: 400 mT/m/msec. Pulse width: 0.32-0.64 msec, oscillating between positive and negative gradients. Rf pulse freq: 126 MHz, frequency modulated for slice position.

The conventional sequence used for fMRI is multi-slice echo planar imaging. In this, the largest gradient is applied as a bi-polar square wave, which is often modified to be more trapezoidal or sinusoidal in form (to smooth the edges). Typically for one image this is applied for 20-100 ms with a fundamental frequency of 2 to 0.5 kHz. One of the other two gradients is usually applied as a series of smaller pulses (100 μs duration typical) at the zero crossings of the big switched gradient, whilst the third (slice select) gradient is generally just applied at the beginning of the sequence as a bi-polar square pulse, typically lasting 3-5 ms. The rf is usually just applied at the same time as the slice select gradient.

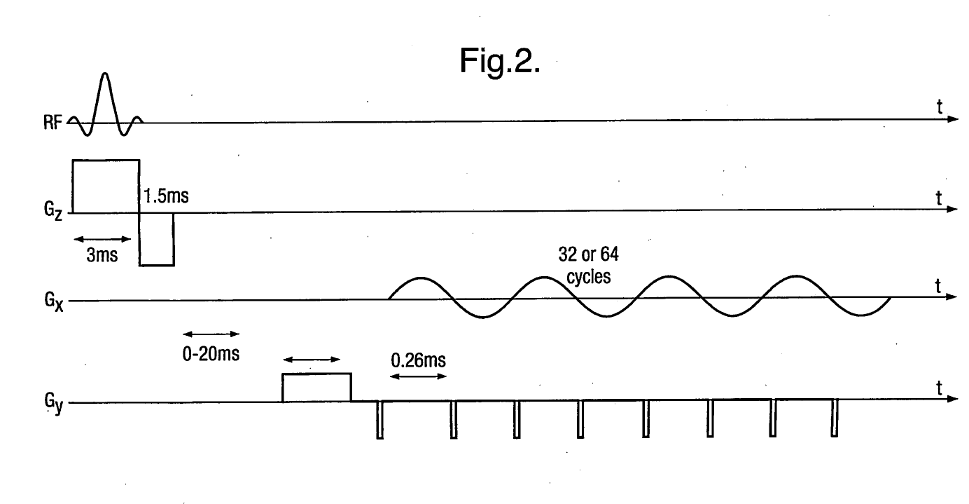

FIG. 2 shows the basic EPI sequence used in the arrangement of FIG. 1. Gz denotes slice select, Gx is the large gradient and Gy is the smaller pulsed gradient. The rf pulses are also shown in FIG. 2. In the tests described further hereinbelow, Gx was on for 30 ms. Depending on the MRI machine used, slice gradient times can vary by a factor of 2, and the switched gradient could be lower by a factor of 2 in frequency and strength.

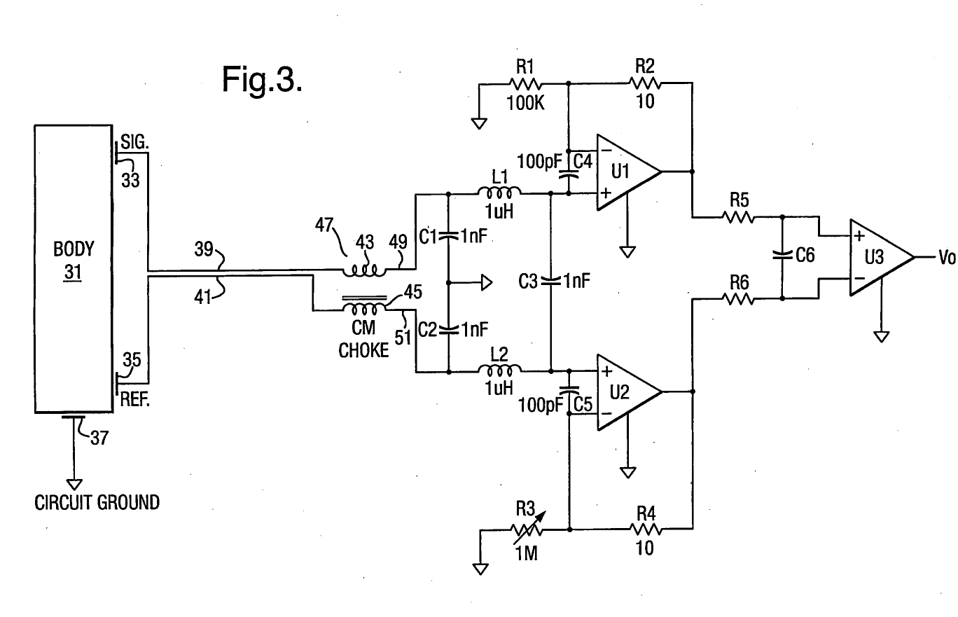

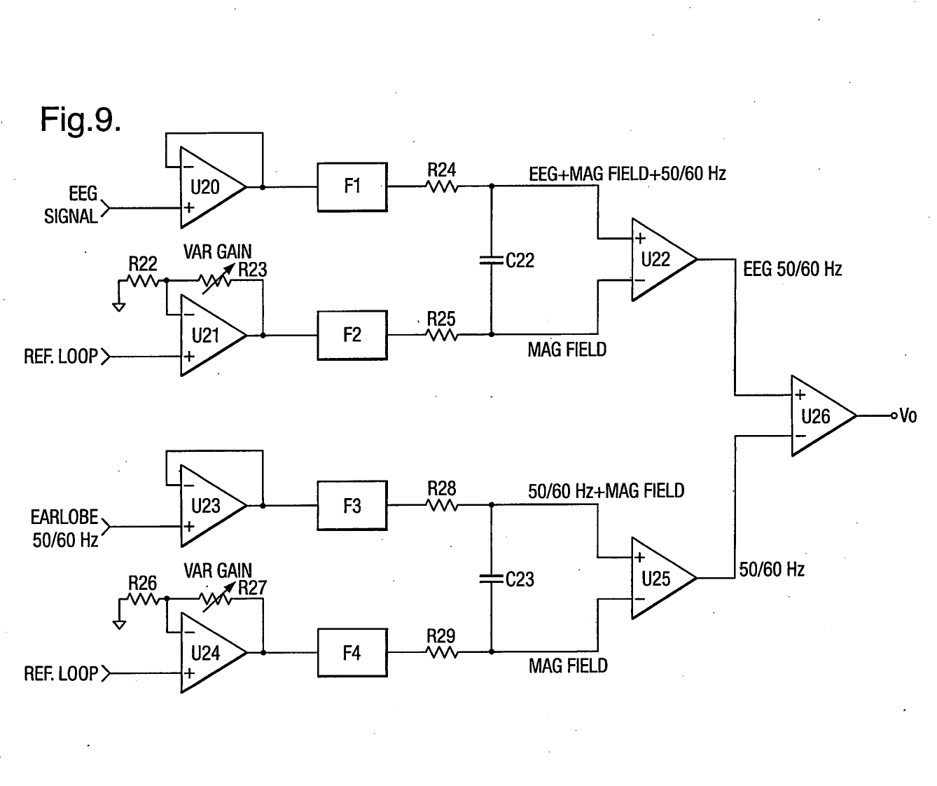

FIGS. 3 to 5 show examples of preamplifier networks for reducing interference for concurrent fMRI and EEG measurements. The preferred embodiments of the invention shown in FIGS. 8 to 21 in which one or more reference electrodes are not in direct electrical contact with the subject are directed to improving the reduction of interference signals over the performance of circuits such as those shown in FIGS. 3 to 5.

FIG. 3 of the accompanying drawings shows a single channel of EEG data acquisition circuitry. It incorporates a reference loop and other means for suppressing interference generated by fMRI. As shown in this figure, attached to a head of a subject 31 are a signal electrode 33, a reference electrode 35 and a circuit ground electrode 37 for biopotential signal acquisition. In order to minimize rf noise in the EEG signal, the electrodes are not metallic, but preferably carbon-loaded material. In order to minimize interference to FMRI, the use of metals, glues, epoxies, etc. should be avoided.

Wires 39 and 41 respectively run from the signal electrode 33 and reference electrode 35 and are physically placed as close together as possible. As the electrode lead wires 39, 41 are made of carbon fiber, wire electrodes can be implemented simply by using the ends of the wires held in place mechanically on the scalp or earlobe and electrically connected to the body 31 with electrode gel. The reference electrode 35 is preferably located on an earlobe, and the wire 41 from the reference electrode 35 is positioned to extend from the reference electrode 35 to a position proximate the signal electrode 33 located on the scalp. The wire 39 connected to signal electrode 33 is then twisted with the wire 41 and the twisted pair, of a length approximately from 2 to 5 meters is connected to filtering and amplification circuitry as described further hereinbelow.

In multi-channel applications comprising a plurality of signal electrodes, each signal electrode wire 39 is paired with a separate wire 41 coming from the reference electrode 35 and all the shielded twisted pairs are bundled together with the ground reference lead wire to form the electrode cable set.

As shown in FIG. 3, at their respective ends remote from the signal 33 and reference 35 electrodes, the shielded twisted pair of wires 39, 41 are connected to respective inputs of the windings 43, 45 of a common mode choke 47. The output terminals 49, 51 of the common mode choke 47 are connected to circuit ground via two capacitors C1 and C2 respectively. Common mode (voltages that are the same for both wires) rf is greatly reduced by the common mode choke 47 in combination with the two capacitors C1 and C2.

The first output terminal 49 of the common mode choke 47 is also connected to the input terminal of a first inductor L1 and the second output terminal 51 of the common mode choke 47 is connected to the input terminal of a second inductor L2. The output terminals of the first and second inductors L1, L2 are bridged by a third capacitor C3. Thus, residual differential mode rf from the output of the common mode choke 47 is thereby converted to common mode by the inductors L1 and L2 respectively connected at one end to choke outputs 49, 51 and at their other ends, bridged by the third capacitor C3. The inductors L1, L2 preferably have an inductance of around 1 pH but ferrite beads having an impedance of several hundred ohms at the relevant rf frequency may be located on the lead wires associated with the inductors L1 and L2. These should be situated sufficiently far from the static magnetic field of the scanner head to avoid saturation. Capacitors C1, C2 and C3 must be small (approximately 1 nF) in order to maintain a high impedance for low frequency signals coming from the signal electrode 33. The output terminal of the first inductor L1 is connected to the non-inverting input of a first operational amplifier U1. A fourth capacitor C4 is connected between the non-inverting and inverting inputs of the first operational amplifier U1. The inverting input of the first operational amplifier U1 is also connected to a first terminal of a first resistor R1. The other terminal of the first resistor R1 is connected to circuit ground. A second resistor R2 is connected between the first terminal of the first resistor R1 and the output of the first operational amplifier U1.

The fourth capacitor C4 may preferably have a capacitance of around 100 pF and the resistors R1 and R2 may have resistances of around 100 Kohms and 100 hms respectively.

Similarly, the output terminal of the second inductor L2 is connected to the non-inverting input of a second operational amplifier U2. A fifth capacitor C5 is connected between the non-inverting and inverting inputs of the second operational amplifier U2 and the inverting input of the second operational amplifier U2 is also connected to a first terminal of a third resistor R3. The other terminal of the third resistor R3 is connected to circuit ground. A fourth resistor R4 is connected between the first terminal of the third resistor R3 and the output of the second operational amplifier U2. The third resistor R3 is preferably a variable resistor having a resistance of around 1 Mohm. The fourth resistor R4 preferably has a resistance of around 10 ohms.

The output of the first operational amplifier U1 is also connected to a first terminal of a fifth resistor R5 and the output of the second operational amplifier U2 is also connected to a first terminal of the sixth resistor R6. The second terminals of the resistors R5 and R6 are connected to the non-inverting and the inverting inputs respectively of a third differential amplifier U3. A sixth capacitor C6 bridges the inverting and non-inverting inputs of the third differential amplifier U3 and the inputs of the third capacitor C3. The output signal Vo of the third differential amplifier U3 is the interference reduced signal.

The “reference loop” is the circuit formed by following the path from the reference electrode 35 through its associated wire 41, into a non-inverting input of an amplifier U2, then to circuit ground leading back to the body through ground electrode 37. An analogous loop is formed in the signal pathway from signal electrode 33 through wire 39 into the non-inverting input of another amplifier U1 and back to the body 31 through circuit-ground and the ground electrode 37.

The first and second low noise operational amplifiers U1 and U2 have a high input impedance with gains approximating one and respectively receive signals at their non-inverting inputs from the inductors L1 and L2. The amplifiers U1 and U2 serve as impedance transformers, presenting a high impedance to the electrodes and a low impedance driving the respective inverting and non-inverting inputs of the third amplifier U3. The gains of amplifiers U1 and U2 are set by the resistors R1 to R4, with R3 being variable to match closely the gains of U1 and U2. Capacitors C4 and C5 are connected between the respective inverting and non-inverting inputs of U1 and U2 to minimise the low frequency response of the amplifiers U1 and U2 caused by rectification of any remaining rf appearing at the inputs. The outputs of U1 and U2 are connected to resistors R5 and R6 respectively in series with respective inverting and non-inverting inputs of the third differential amplifier U3. These combine with a capacitor C6 (across the inputs of U3) to convert differential mode voltages above a set −3 dB (filter cutoff) frequency to common mode voltages. U3 is preferably a high speed differential amplifier (such as Analog Devices™ AD 8129) which is capable of rejecting common mode voltages up to rf.

Thus in combination, R5, R6, C6 and U3 function as a single pole low pass filter, converting differential mode voltages on the signal and reference lines to common mode voltages on a −6 dB per decade basis above the −3 dB cutoff frequency. Such a filter converting differential mode voltages to common mode voltages is hereinafter referred to as a DM/CM filter. U3 also performs subtraction of the reference voltage from the signal voltage in the bandwidths below the DM/CM filter cutoff frequency. Any mismatch between interference voltages in the signal and reference lines below the DM/CM cutoff frequency, results in a residual interference component in the signals. Above the cutoff frequency, both signal and reference signals are filtered but as the filter is only single pole, any large mismatches in noise voltage appearing on the signal and reference lines at frequencies near the filter cutoff, will result in residual interference appearing at the output.

The DM/CM filter cutoff frequency is set as low as possible to obtain maximum rejection of magnetically induced interference voltages. Typically, R5 and R6 may be 365Ω and C6 may be 1.0 μF, resulting in a −3 dB cutoff frequency of approximately 218 Hz. U3 amplifies the remaining differential mode signal received from U1 and U2 by a gain of 10, and this output is further amplified and filtered with high pass and low pass filters (not shown). A typical filter implementation includes a single pole high pass filter with a −3 dB frequency of 1.0 Hz and a 4-pole Butterworth low pass filter with a −3 dB frequency of 256 Hz. The combination of all filters results in a final signal bandwidth, which may preferably be from 1 to 100 Hz. To reduce interference still further, the bandwidth may be narrowed, depending upon the frequency range of the signal of interest.

The large magnetic fields of fMRI can induce voltages in the order of volts in the reference loop and its analogous loop. The induced voltages are reduced by minimizing the areas of the loops, but the physical arrangement of electrodes on the scalp versus the site of the ground electrode results in a loop that cannot be avoided and is large enough to result in large induced voltages. As the reference voltage is subtracted from the signal voltage, spatially associating the signal and reference loops in close proximity results in further reduction of the induced interference. A single wire from the reference electrode for all of the signal channels may be used which results in large spatial mismatching for most channels. With the arrangement shown in FIG. 3, a plurality of signal electrodes, each with its own signal wire will preferably be used. A separate reference wire will then be employed for each signal channel, closely following the signal lead wire (preferably twisting the wires) so that spatial matching of the loops is maximized. All of the reference wires terminate electrically at the reference electrode 35 or reference electrodes, if more than one of the latter is provided. This means that in such an arrangement, many reference wires terminate on a single reference electrode 35 or group of reference electrodes.

Benefits of the circuit of FIG. 3 are provided by the use of separate wires from the reference electrode 35 for each signal wire (reference loops), the common mode choke 47, and the combination of gain-matched buffer amplifiers U1 and U2, DM/CM filter and a high speed differential amplifier U3. Using the ends of carbon lead wires as electrodes and the use of a second shield, connected to circuit ground and surrounding the twisted pairs of wires, is also advantageous.

The fundamental object of the circuit shown in FIG. 3, is to reduce interference voltages to low levels and amplify the signal. This has to be achieved across the wide range of frequencies involved. For attenuation of power mains interference, the high impedance of the buffer amplifiers U1 and U2, tight gain matching, the high common mode rejection of U3, tight matching of reference loops and electrical isolation of circuit ground from power or true ground are sufficiently effective. Driving a second twisted-pair shield within the grounded shield, with a common mode signal derived from the signal and reference lines, helps to maintain a high input impedance when using long electrode leads, especially when the twisted pairs of wires are located within a shield connected to the circuit ground. For interference from fMRI magnetic fields, tightly matched reference loops significantly reduce induced voltages, and the R5-R6-C6-U3, DM/CM low pass filter in combination with the 4-pole low pass filter removes most remaining interference. The use of carbon wires, a cable shield connected to circuit ground, rf common mode and differential mode filters, rf shunt capacitors C1 and C2 across the buffer amplifier U1 and U2 inputs and the high speed differential amplifier U3 also act in combination to reduce rf interference.

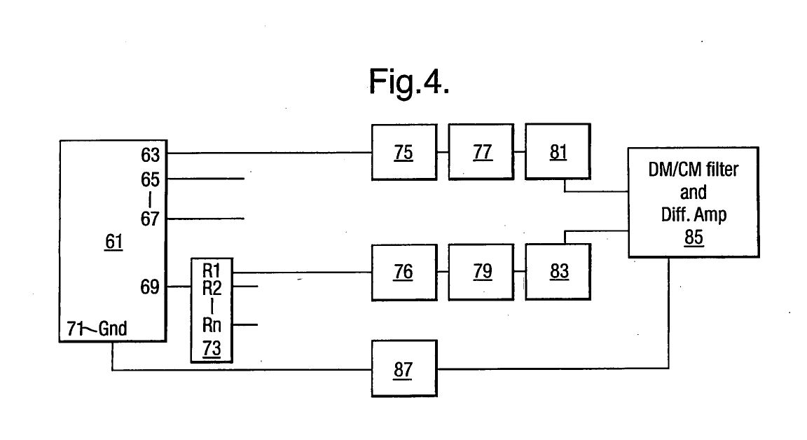

Another circuit is depicted in FIG. 4, in which numeral 61 represents the subject, with signal electrodes 63, 65, etc (typically attached to the scalp), a compensation electrode 69 (typically attached to the earlobe), and ground electrode 71. The electrodes 63 to 71 and connecting wires are typically carbon loaded material (to lower conductivity thus reducing rf currents in the electrodes and wires), with a 10KΩ to 15KΩ carbon resistor (not shown) inserted in line near the electrodes for rf current-limiting, safety and filtering. Numeral 73 represents a conductive junction (typically of carbon loaded material for rf current reduction) for distributing a multitude of reference wires R1 to Rn which may also be formed of carbon loaded material, each of which is placed in close proximity to, and where possible, twisted with, a signal electrode wire. The compensation electrode 69 is preferably attachable to an earlobe of the subject. Each reference wire forms a reference loop that is closely matched to the loop formed by the signal (or compensation) electrode wire.

Each signal-reference wire pair 63/R1 to Rn etc is connected via rf filters 75, 76 etc to a respective pair of preamplifiers 77, 79 etc. At the input of the preamplifiers 77, 79 etc, additional rf filtering may be implemented by using a common mode choke across the wire pairs followed by capacitors to isolated ground, and a series indicator (1 μH typically) or a ferrite chip (presenting an rf impedance of several hundred ohms) in each line followed by a capacitor (typically 1 nF) across the lines as in the arrangement of FIG. 3. An rf filter 87 consisting of a series inductor (1 μH typically, or a ferrite chip) followed by a capacitor to ground (1 nF typically) is also placed in the ground line. The output of each preamplifier is connected to a low pass filter. These are denoted as low pass filters 81, 83 etc for the preamplifier pair 77, 79. Thus, each signal line from a signal electrode 63 and each reference line Rn associated therewith is connected to its own rf filter, with the outputs of the low pass filters connected to a circuit unit (denoted as DM/CM filter and Diff Amp 85). The circuit unit performs filtering and subtracting functions in a manner similar to that of FIG. 3.

The signal and reference wire pairs are bundled together along with the ground electrode wire for about from 2 to 5 meters typically, at which point the carbon wires are terminated inside a shielded metal (aluminium) enclosure containing rf filters 75 etc for each wire (only one is shown for electrode 63 for simplicity in the diagram). The metal case of the rf filter enclosure is bonded to the frame of an MRI apparatus for establishing a low impedance rf ground. The rf filters consist of series inductors (typically 1 μH) followed by a capacitor connected to an isolated rf ground in the enclosure, which is connected, in turn, to the metal case by a single 1 nF capacitor. Metallic (usually copper) wires in twisted pairs are connected to the outputs of the rf filters for each signal-reference pair, and a single metal wire is connected to the ground electrode rf filter output, with the resulting cable bundled inside a metallic shield (shield connected to ground at the rf filter box). This cable is run (typically 2 meters) to a metallic (aluminium) enclosure containing preamplifiers, filters, differential amplifiers, filters, main amplifiers, sample-and-holds, digitizer, digital control and ethernet interface circuitry. The shield of the cable from the rf filter box terminates on the metal casing of the amplifier/digitizer enclosure.

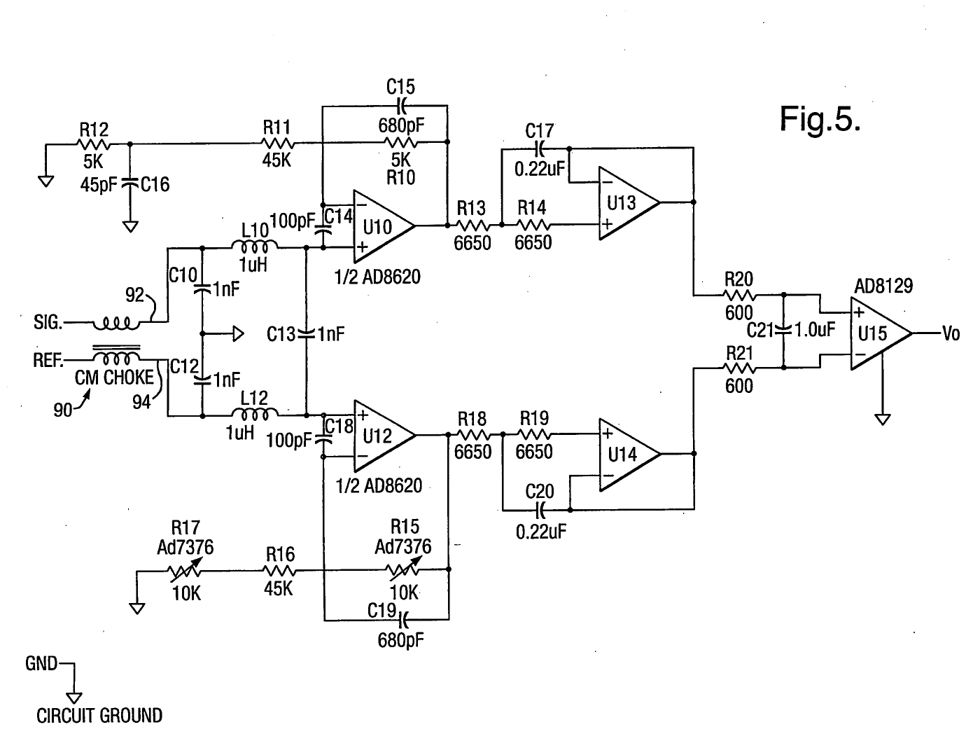

FIG. 5 shows a circuit diagram of the components 75 to 85 in the block diagram of FIG. 4.

The signal and reference leads from the signal and reference electrodes are connected to a common mode choke 90 comprising two windings on a common core. The output 92 of the signal winding of the common mode choke is connected to an RF filter comprising a first capacitor C10 and a first inductor L1. The other terminal of the capacitor C10 is connected to circuit ground. The first terminal of the capacitor C10 is also connected to a first terminal of the first inductor L10 and the second terminal of the inductor L10 is connected to the non-inverting input of a first operational amplifier U10.

The output 94 of the reference winding of the common mode choke 90 is connected to a second RF filter comprising a second capacitor C12 and a second inductor L12. The reference winding is connected to the first terminal of the second capacitor C12 and the second terminal of capacitor C12 is connected to circuit ground. The first terminal of the capacitor C12 is also connected to the first terminal of the second inductor L12 and the second terminal of the inductor L12 is connected to the non-inverting input of a second operational amplifier U12.

A third capacitor C13 is connected between the non-inverting inputs of the operational amplifiers U10 and U12. A further capacitor C14 is connected between the non-inverting and inverting inputs of the first operational amplifier U10. Feedback components comprising a resistor R10 and a capacitor C15 are connected in parallel with each other between the inverting input and output of the first operational amplifier U10. A first terminal of a further resistor R11 is connected to the inverting input of the operational amplifier U10 and the second terminal of the resistor R11 is connected to the first terminal of a further resistor R12. The first terminal of the resistor R12 is also connected to the first terminal of a further capacitor C16. The second terminal of the resistor R12 is connected to circuit ground as is the second terminal of the capacitor C16. The capacitor C16 is thereby connected in parallel with the resistor R12.

The output of the operational amplifier U10 is connected to a first terminal of a resistor R13 and the second terminal of the resistor R13 is connected to a first terminal of a further resistor R14 and is also connected to the first terminal of a further capacitor C17. The second terminal of the resistor R14 is connected to the non-inverting input of an operational amplifier U13. The second terminal of the capacitor C17 is connected to the inverting input of the operational amplifier U13 and to the output of the operational amplifier U13.

A further capacitor C18 is connected between the inputs of the second operational amplifier U12. Feedback components comprising a resistor R15 and a capacitor C19 are connected in parallel with each other between the inverting input and the output of the second operational amplifier U12. The resistor R15 is preferably a digitally controlled variable resistor.

The resistor R15 is connected in series with two further resistors R16 and R17, the second terminal of the resistor R17 being connected to circuit ground. R17 is preferably a digitally controlled variable resistor.

The output of the second operational amplifier U12 is also connected to the first terminal of a further resistor R18, the second terminal of the further resistor R18 being connected to the first terminal of a resistor R19 and to the first terminal of a capacitor C20. The second terminal of the resistor R19 is connected to the non-inverting input of a further operational amplifier U14 and the second terminal of the capacitor C20 is connected to the inverting input of the operational amplifier U14 and to the output of the operational amplifier U14.

The output of the third operational amplifier U13 is connected to the first terminal of a resistor R20, the second terminal of the resistor R20 being connected to the non-inverting input of a fifth operational amplifier U15.

The output of the operational amplifier U14 is connected to the first terminal of a resistor R21 and the second terminal of the resistor R21 is connected to the inverting input of the fifth operational amplifier U15.

A further capacitor C21 is connected between the inverting and non-inverting inputs of the fifth operational amplifier U15 between the second terminals of the resistors R20 and R21. The output of the operational amplifier U15 comprises the interference reduced output voltage V0. The ground connection of the operational amplifier U15 is connected to circuit ground.

The preamplifiers for a signal reference pair are preferably Bi-FET, JFET or CMOS operational amplifiers U10 and U12, with low noise and high input impedance. U10 and U12 may be implemented in the form of a dual operation amplifier integrated circuit such as Analog Devices AD8620 or OP2177. A capacitor (C14 and C18, typically 100 pF) may be connected across the inverting and non-inverting inputs of the operational amplifiers to minimize low frequency response at the op amp output caused by rectification of residual rf at the inputs.

The preamplifiers have a gain of approximately 1 to 2, and serve primarily as impedance transformers to compensate for the relatively high impedance of the electrode-tissue interface. Each signal preamplifier U10 has a fixed gain, while the reference preamp may have a variable gain (adjusted by varying feedback resistance around the operational amplifier using digitally controlled resistors R17 and R15), which allows dynamic trimming of the reference voltage amplitude, to provide a better match of interference voltages on the signal and reference lines for subsequent subtraction. R17 and R15 may be implemented with Analog Devices AD7376 digital potentiometers with 10KΩ resistance. In the circuit implementation shown in FIG. 5, the gain of the signal preamplifier is 1.1 and the reference preamplifier gain varies from 1.0 to 1.2. Wider ranges may be used by setting the gain of the signal preamplifier to the centre of the range (for example, center gain of 2.0) and varying the reference preamplifier gain between the edges of the range (for example, a range of from 1.0 to 4.0).

Since the digital potentiometers present a capacitance in addition to a resistance to the preamplifier feedback circuit, compensation capacitors C15 and C16 (typically 680 pF for the AD7376) are added to the feedback loops of the preamplifiers. C16 (typically 45 pF) is used in the signal preamplifier feedback network as shown, to match a capacitance added by R17 in the reference preamplifier feedback network, in order to maintain similar frequency responses for the preamplifiers.

A second order low pass filter 81, 83 etc (preferably of the Bessel type to minimize pulse overshoot) with a gain of one follows each preamplifer. As shown in FIG. 5, operational amplifiers U13, U14 (AD 8620, OP 2177 or similar) and circuit elements R13, R14, R18 and R19 and C17, C20, construct second order Bessel filters with a cutoff frequency of 145.4 Hz. The resulting filtered signal and reference voltages are input via a first order DM/CM filter, to wide bandwidth differential amplifier U15 (with a typical gain of about 10), for the purpose of filtering both signal and reference lines, and also, subtracting the reference from the signal in the bandwidth below the filter cutoff frequency. However, with correct selection of cutoff frequencies for the Bessel and DM/CM filters, a third order low pass filter may be realised at the output of the differential amplifier, instead of a single order filter. Thus, better filtering of the interference is achieved. In FIG. 5, circuit elements R20, R21 and C21 in combination with U15 (Analog Devices AD 8129 or similar) form a DM/CM filter with a −3 dB frequency of 132.8 Hz. The resulting third order filter has a −3 dB cutoff frequency of 100 Hz. Following the differential amplifier, additional stages of amplification and low pass filtering are employed, as usually practiced in the acquisition of EEG. The ground electrode lead (after rf filtering (not shown)) is connected to isolated circuit ground. Isolation is held to approximately 1 nF in order to allow low impedance for rf filtering yet maintain high impedance for low frequency interference rejection and patient safety.

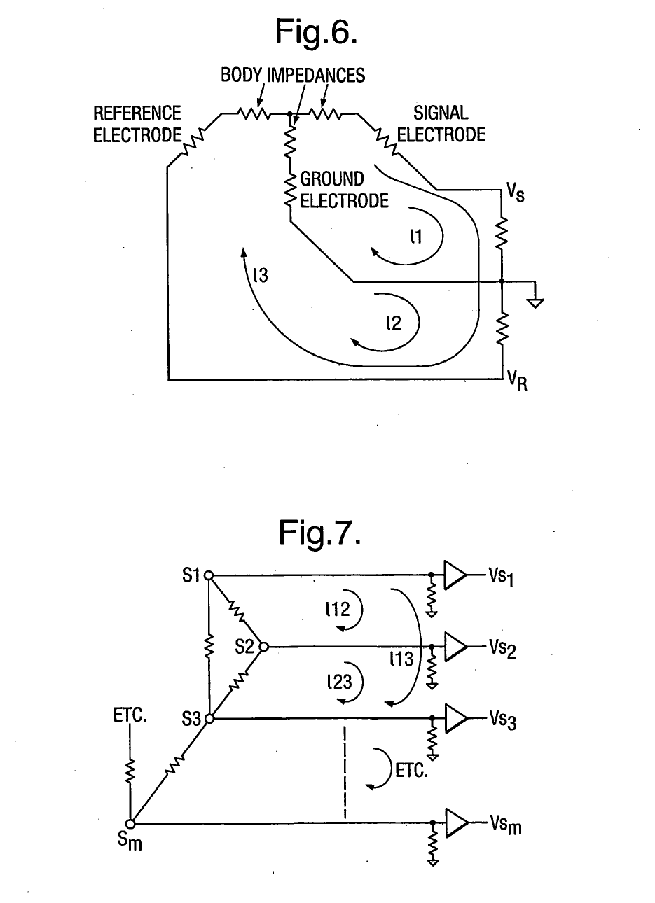

FIG. 6 shows an equivalent circuit for the reference loop arrangement of a single channel for use in the circuits shown and described in respect of FIGS. 3 to 5. As shown in FIG. 6 there are three loops formed by the circuits of FIGS. 3 to 5 that comprise the signal, reference and ground electrodes and associated wires and impedances.

The contact between a wire and the body of a subject has an intrinsic associated impedance and as the leads to the electrodes may be formed of a material such as carbon fibre, the leads may have an intrinsic resistance in addition to any resistance added for safety reasons. FIG. 6 shows three impedances representing the contacts of the signal, reference and ground electrodes and the subject body, these contacts having a common point representing the actual body of the subject. The impedances of the leads together with any additional resistors are shown lumped together as an electrode impedance. The leads from the signal and reference electrodes are returned to circuit ground and thus the ground electrode at the inputs of the amplifiers, the inputs of the amplifiers having an effective impedance.

The signal electrode loop 11 comprises the impedance between the electrode and the body, the signal lead, the input impedance of the amplifier, the ground electrode lead and the body impedance from the ground electrode to the body. The reference electrode loop 12 comprises the body impedance from the reference electrode to the body, the reference lead, the input impedance of the amplifier to ground, the ground lead and the impedance between the ground electrode and body.

The third loop 13 comprises the impedance between the signal electrode and the body, the signal electrode lead, the input impedance of the amplifier to circuit ground, the input impedance of the reference input, the reference electrode lead and the impedance of the reference electrode to the body.

External varying magnetic fields passing through the area formed by the loops could induce unwanted voltages in the circuit which obscure the desired signal voltages detected on the body. However, the interference voltages are reduced by minimizing the area formed by the loops, and may also be reduced by subtracting the voltage appearing on the reference circuit from the voltage on the signal circuit, since with appropriate spatial arrangement, there should be no physiological signal of interest in the reference circuit. In the equivalent circuit of FIG. 6, if the areas formed by loops 11 and 12 are well-matched, subtracting the reference voltage Vr from signal voltage Vs will significantly reduce or cancel the magnetically induced interference induced in the signal channel due to loop 11. However, a third loop 13 may be formed via the low impedance of the body and electrodes, since the reference loop is connected to an earlobe. However, the interference induced in loop 13 may be minimized by reducing the loop area.

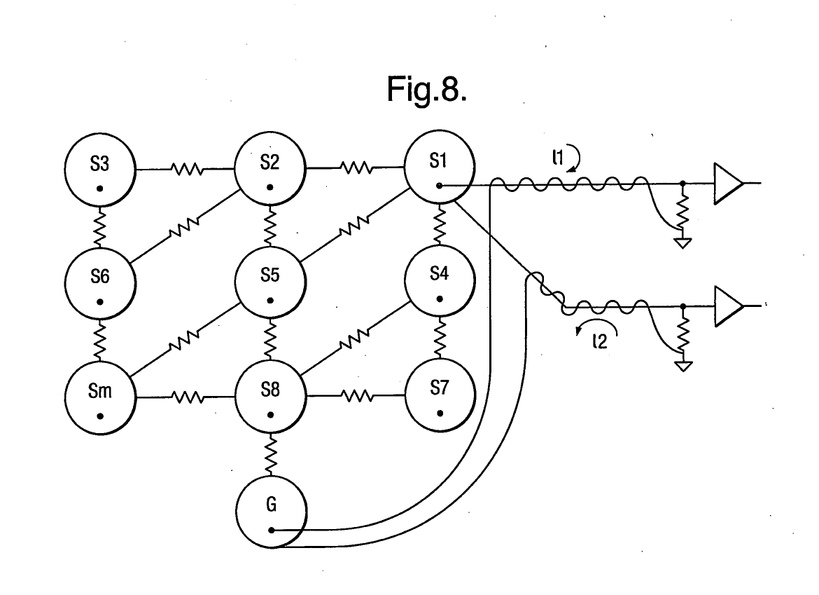

FIG. 7 shows an equivalent circuit for demonstrating another source of interference which may not be so well reduced by the circuit arrangements of FIGS. 1-5. As depicted, all signal leads (S1, S2, . . . Sm) are connected via the impedances of the electrodes and body (shown as single resistors between various signal electrode sites), thus forming loops (I12, I13, I23 etc). A parallel pathway for the reference loop circuit that is well-matched to each signal-signal loop is required in order to cancel the magnetically induced interference by subtraction of the reference loop voltage. The arrangements of FIGS. 1-5 effectively only provide a single reference loop for each signal channel, but that reference loop does not match the additional loops formed by the multitude of signal channels, as shown in FIG. 7.

Each signal site is assumed to be connected to all other signal sites (and ground electrode) via electrode and body impedances as depicted in FIG. 7.

Preferred embodiments of the present invention recognise that the interference induced in loop I3 shown in FIG. 6 may effectively be eliminated by removing the reference lead from connection to the earlobe, and providing a separate ground return added to complete the circuit for loop I2. In this case, the loops I1 and I2 of the equivalent circuit of FIG. 6 are thus physically well matched and smaller in area since each signal and reference circuit has a tightly twisted return lead. As there is no longer a low impedance pathway between the reference network and the signal circuit, loop I3 is broken, thus drastically reducing interference from that source.

In a first embodiment of the invention, to provide a better match for the totality of the loops in each signal channel, an isolated reference network or mesh may be used instead of the reference electrode. FIG. 8 is an equivalent circuit showing a section of such an arrangement in which multiple signal electrodes S1 to Sn may be mounted on the body with an accompanying reference loop network or mesh denoted by rings around each signal electrode which in turn are denoted by a dot within the ring.

For clarity, a single channel of signal and reference outputs is shown, the ring around each signal electrode representing a point adjacent the signal electrode from which the reference contact is taken. However, all such points are interconnected through the mesh and this represented in FIG. 8 by resistors linking the rings.

The ground electrode (designated by “G”) is also surrounded by the mesh. A lead wire from the ground electrode is twisted with the lead wire from the signal electrode and a lead wire from the reference mesh at a point adjacent to the ground electrode is twisted with the lead wire from the reference point corresponding to the signal electrode. The mesh extends around the ground electrode.

As can be seen in FIG. 8, any pathway between signal electrodes is closely matched by a reference pathway formed by the conductive reference network. To obtain the best match of induced voltages in the loops, the impedances of the pathways in the signal and reference loops should be similar.

Preferred embodiments of the invention based on the equivalent circuit shown in FIG. 8 preferably utilise a mesh of carbon (or similar) wires or a preformed conductive fabric mesh located in the area of the signal electrodes (such as mounted on an electrode cap, insulated from the body) to provide a multitude of pathways for reference loops to match signal circuit loops. Further, these embodiments eliminate the third loop formed between the signal and reference wires, by virtue of isolating the reference circuits from the body, i.e., the reference leads are no longer connected to the earlobe. Further, the improved method provides a means of rejecting mains power interference by means of a separate signal circuit (with an isolated parallel reference loop) connected to the earlobe, subsequently subtracted from the EEG signal channels (not shown in FIG. 8), as described below.

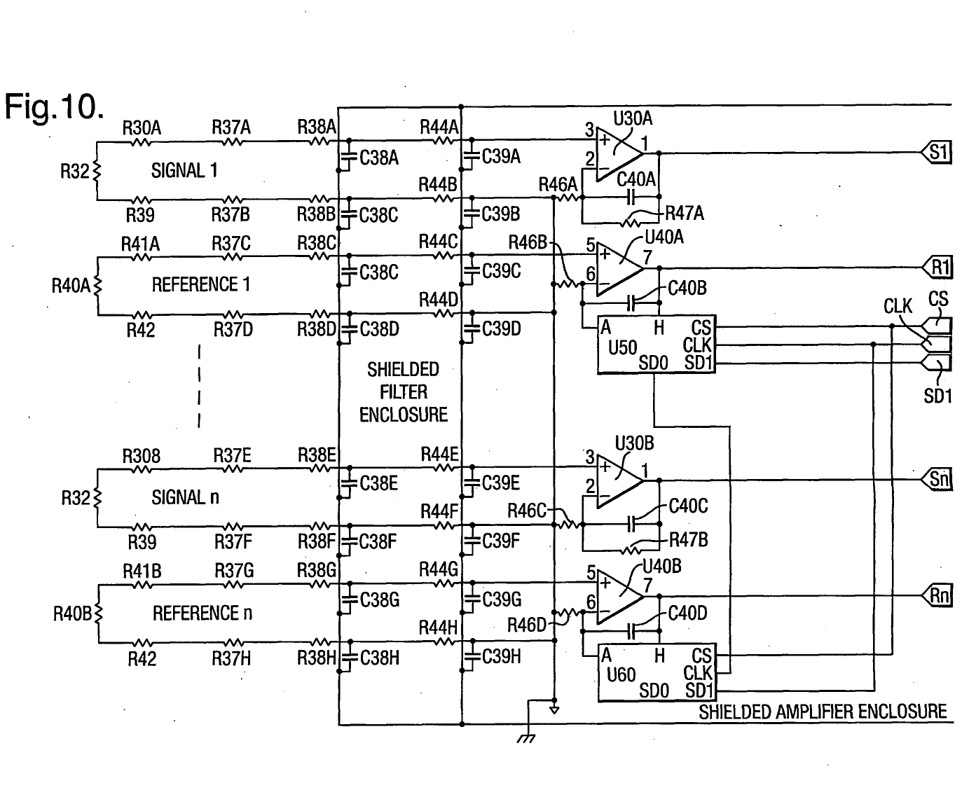

FIG. 9 shows part of an actual circuit comprising amplifiers and filters associated with a single channel of EEG for implementing the principles embodied in the equivalent circuit of FIG. 8. The signal wire is connected to the non inverting input of an amplifier U20, and the reference loop associated with the signal line is connected to the non-inverting input of an amplifier U21. The inverting input of amplifier U20 is connected to the output of the amplifier U20. The inverting input of U21 is connected to the first terminal of a resistor R22 and the second terminal of the resistor R22 is connected to circuit ground. The inverting input of U21 is also connected to a first terminal of a resistor R23, the second terminal of the resistor R23 being connected to the output of the amplifier U21. The resistor R23 is preferably a digital potentiometer.

The amplifier U20 is a high impedance low noise operational amplifier with fixed gain of 1 to 2. Amplifier U21 is also a high impedance low noise operational amplifier.

The gain of the amplifier U21 is controlled by the digital potentiometer R23 allowing dynamic setting of the gain of U21 by software control for the purpose of matching the amplitude of induced interference voltage in the reference loop circuit with the induced interference voltage in the signal circuit. Alternatively, the gain of U21 may be matched to that of U20 by closely matching (to within 5% or less) the gain-setting components of the amplifiers.

The output of the amplifier U20 is connected to the input of a filter F1 and the output of the amplifier U21 is connected to the input of a filter F2. The filters F1 and F2 are matched 2-pole low pass active filters with low overshoot characteristics such as a Bessel filter. The output of the filter F1 is connected to the first terminal of a resistor R24 and the second terminal of the resistor R24 is connected to the first terminal of a capacitor C22 and to the non-inverting input of a further amplifier U22. The output of the filter F2 is connected to a first terminal of a resistor R25, the second terminal of the resistor R25 being connected to the second terminal of the capacitor C22 and to the inverting input terminal of the amplifier U22.

The resistors R24 and R25 and the capacitor C22 form a low pass filter in combination with the differential amplifier U22, which preferably maintains high common mode rejection at high frequency (for example, the AD8129 differential amplifier manufactured by Analog Devices, Inc., with a common mode rejection of 90 dB at 1 MHz).

The output of U22 is the desired signal with a gain of 10, minus the matched interference of the reference loop. Any mismatched interference in the signal and ref loops below the cutoff frequency of the low pass filters will be present. Mains powerline interference is also present at the output of U22. A means of reducing powerline interference in the signal is implemented by connecting a signal channel with accompanying reference loop to an earlobe or scalp site close to an ear to form a compensation loop.

The signal from the earlobe (consisting primarily of induced powerline interference voltages from the human body) is connected to the non-inverting input of a further operational amplifier U23. The inverting input of the operational amplifier U23 is connected to the output of the operational amplifier U23 and the output of the operational amplifier U23 is connected to the input of a filter F3.

The input from the associated reference signal is connected to the non-inverting input of another operational amplifier U24. The inverting input of the operational amplifier U24 is connected to a first terminal of a resistor R26, the second terminal of the resistor R26 being connected to circuit ground. The non-inverting input is also connected to the first terminal of a resistor R27, the second terminal of the resistor R27 being connected to the output of the operational amplifier U24 and to the input of a further filter F4. The resistor R27 is preferably a variable resistor.

The output of the filter F3 is connected to the first terminal of a resistor R28 and the second terminal of the resistor R28 is connected to the first terminal of a capacitor C23 and to the non-inverting input of a further amplifier U25. The output of the filter F4 is connected to a first terminal of a resistor R29, the second terminal of the resistor R29 being connected to the second terminal of the capacitor C23 and to the inverting input terminal of the amplifier U25.

The output signal of the amplifier U22 which comprises the EEG signal plus any 50 or 60 Hz interference induced in the electrode leads is passed to the non-inverting input of a differential amplifier U26. The output signal of the operational amplifier U25 which comprises the 50 or 60 Hz signals is passed to the inverting input of the differential amplifier U26. The differential amplifier U26 subtracts the 50 or 60 Hz signal from the EEG plus 50/60 Hz signal to give an output voltage Vₒ comprising the EEG signal.

The amplifier U23 is a high impedance low noise operational amplifier with fixed gain of 1 to 2. Amplifier U24 is also a high impedance low noise operational amplifier.

The gain of the amplifier U24 is controlled by the digital potentiometer R27 allowing dynamic setting of the gain of U24 by software control for the purpose of matching the amplitude of induced interference voltage in the reference loop circuit with the induced interference voltage in the signal circuit.

The filters F3 and F4 are matched filters similar to F1 and F2, and R28, R29 and C23 in combination with U25 (same type of differential amplifier as U3) form a low pass filter. U25 has a variable gain function implemented by means of a digital potentiometer under software control. The output of U25 is the powerline interference voltage minus the matched magnetic interference from the reference loop.

Amplifier U26 typically has a gain of 50, and the output is the amplified EEG signal with significant amounts of interference from magnetic (FMRI) and electrostatic (AC power) sources removed. Further amplification and filtering of the EEG may be implemented on the output of U26.

FIG. 9 thus shows a single channel implementation of the improved reference loop in a multi-channel implementation the output of U25 is fed to the inverting inputs of the equivalent U26 amplifiers for all the EEG signal channels.

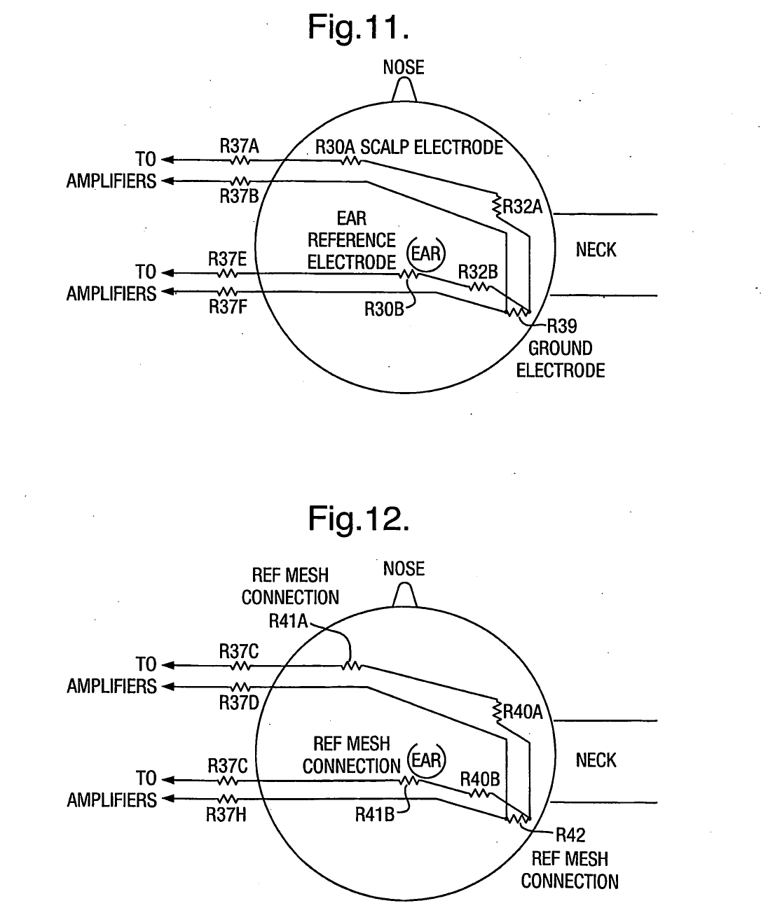

Another embodiment exemplifying apparatus and a method according to the present invention is shown in FIGS. 10-16.

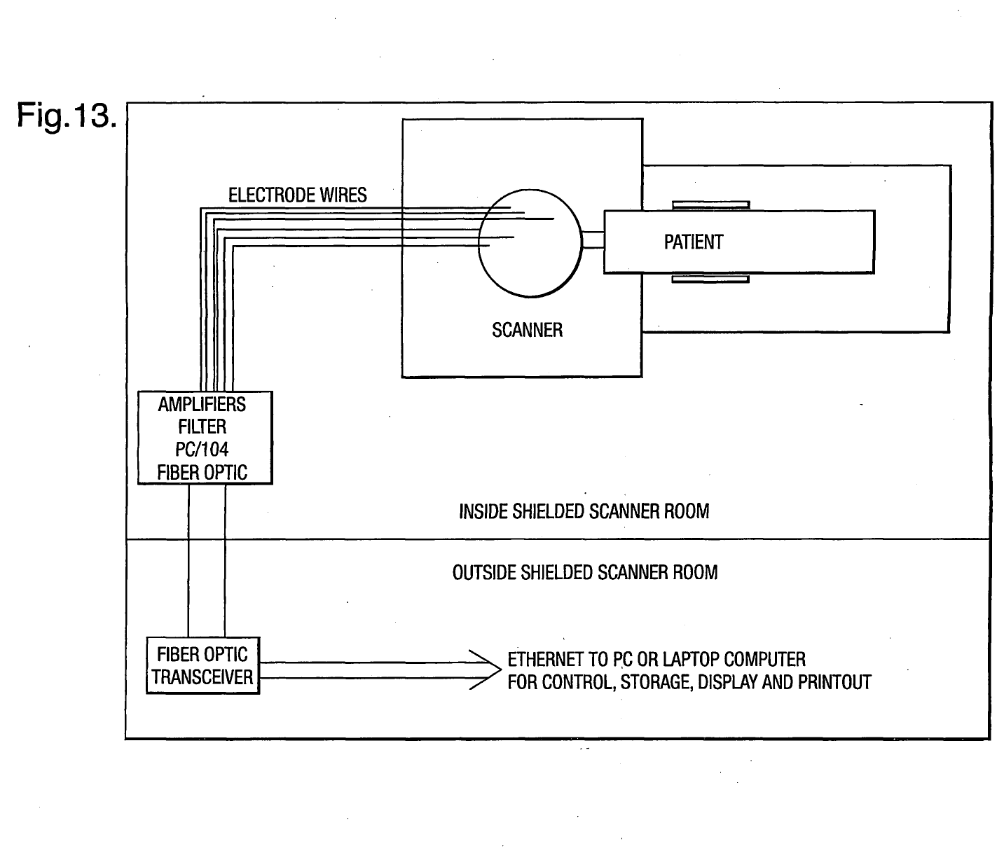

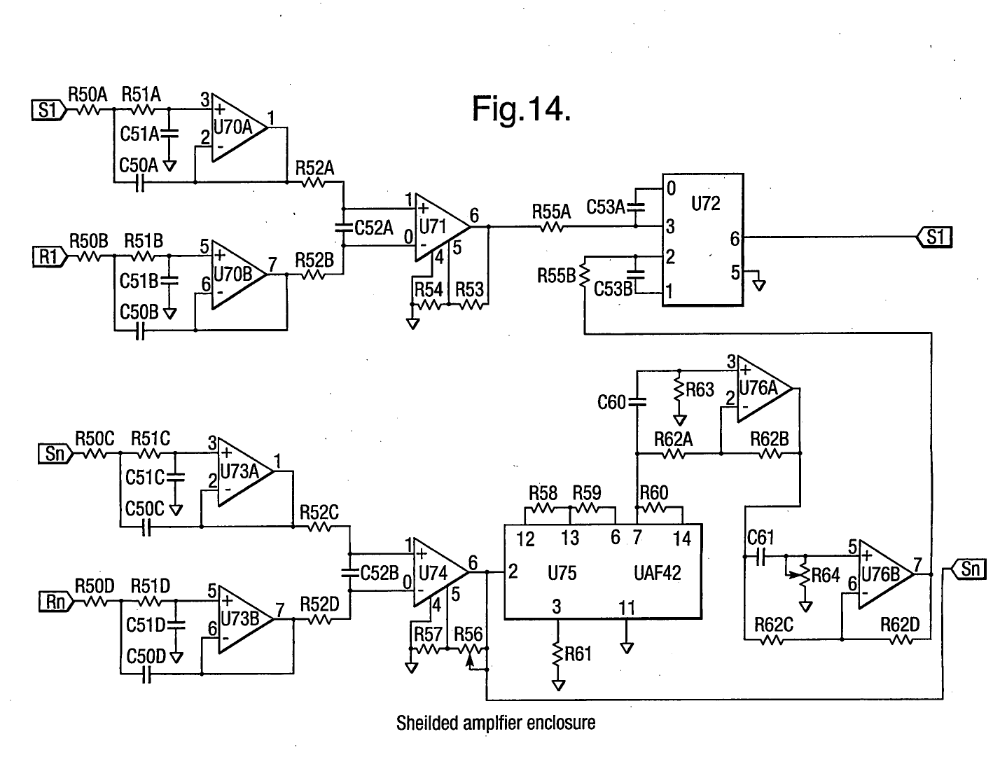

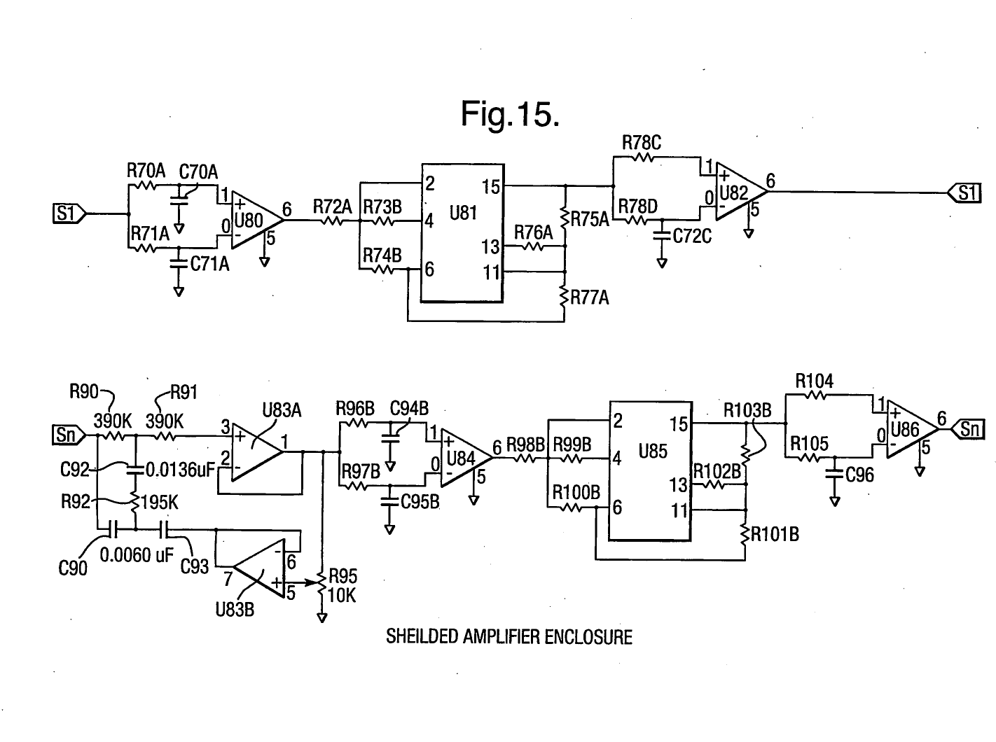

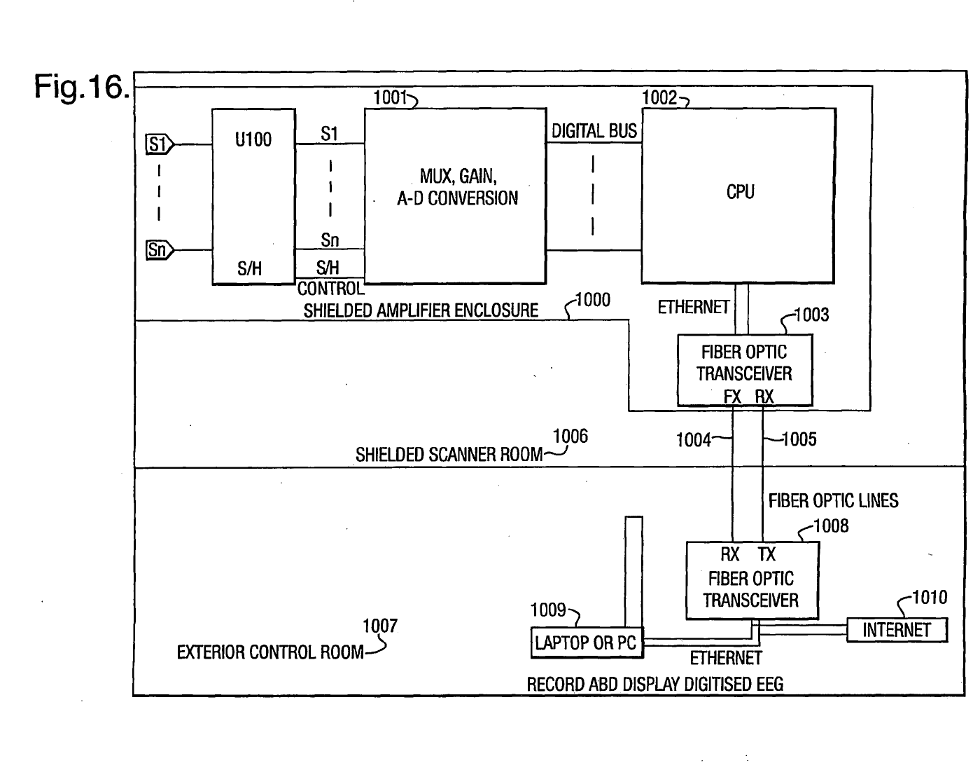

FIG. 10 shows the front end circuitry of this embodiment, which circuitry is attached to signal, reference and ground electrodes, which are attached to the subject who is inside the scan head within the scan room. FIGS. 11 and 12 show the electrode connections to the subject's head and the connections of the reference mesh, respectively. FIG. 13 shows the location of subject and system components with respect to the scan room. FIGS. 14, 15 and 16 show other circuitry details of this embodiment.

Referring to FIG. 10, there are n measurement channels, where n ranges typically from 2 to 1024. For convenience, only the 1^{st} and n'th channels are actually shown in the drawing. Each measurement channel comprises a signal line and a reference line. The signal line and reference line of each channel are paired with a respective ground line (not shown).

Thus, as shown, there are n measurement channels (1 to n) of identical construction such as is shown for measurement channel 1. As the n channels are of identical construction, only Channel 1 will be described in detail below. Channel 1 comprises signal line pair designated “Signal 1” and reference line pair “Reference 1”. As depicted, the signal line of “Signal 1” is connected to the scalp for EEG via a signal or measurement electrode with an impedance represented by resistor R31A, preferably having an electrode impedance of around 10K ohms or less. Other signal electrodes are denoted R30B etc. All body electrodes preferably are constructed of a resistive material such as carbon-loaded plastic, or the bare ends of carbon wire. Contact to the body is made via a conductive paste.

In a signal channel 1, outside a shielded filter enclosure, a number of resistors R30A, R32, R37A, R37B, R38A, R38B and R39 are connected in series. A first terminal of the resistor R32 is connected to a first terminal of the resistor R30A and the second terminal of the resistor R30A is connected to the first terminal of the resistor R37A, the second terminal of the further resistor R37A being connected to the first terminal of the resistor R38A. The second terminal of the resistor R32 is connected to the first terminal of the resistor R39 and the second terminal of the resistor R39 is connected to the first terminal of the resistor R37B, the second terminal of the resistor R37B being connected to the first terminal of the resistor R38B. In the reference channel 1, outside a shielded filter enclosure, a number of resistors R37C, R37D, R38C, R38D, R40A, R41A and R42 are connected in series. The first terminal of a first resistor R40A is connected to the first terminal of the resistor R41A, the second terminal of the resistor R41A being connected to the first terminal of the resistor R37C. The second terminal of the further resistor R37C is connected to the first terminal of the resistor R38C and the second terminal of the resistor R40A is connected to the first terminal of a resistor R42. The second terminal of the resistor R42 is connected to the first terminal of the resistor R37D and the second terminal of the resistor R37D is connected to the first terminal of the resistor R38D.

Similar connections exist for the other channel/reference pairs.

For channel 1 (and similarly for all signal channels), the wires represented by R37A and R37B are twisted together tightly to minimize the loop area formed by the wires and hence minimize induced magnetic field interference in the signal.

Thus, in measurement channel 1, R41A is a connection of a carbon wire to a conductive reference mesh that spans the surface of the head but is not in electrical contact with the body. R41A is located very close to R30A. R40A represents the impedance of the reference mesh. R42 is the connection from the mesh to the return wire for the reference loop, represented by R37D. R42 is located very close to R32. The wires for the reference loop (R37C and R37D) are twisted together tightly to minimize loop area, and the pair is twisted together with the R37A-R37B pair to match the paths followed by the loops.

Preferably the impedances of R30A and R41A are matched, as well as those of R32 with R40A, and R39 with R42. However, it is acceptable if only the sums of impedances R30A+R32+R39 and R41A+R40A+R42 are reasonably matched.

In the shielded filter enclosure, in the signal line the second terminal of the resistor R38A is connected to a capacitor C38A and also to the first terminal of a resistor R44A. The second terminal of the resistor R38B is connected to the first terminal of a capacitor C38B and also to a resistor R44B. The second terminals of the capacitors C38A and C38B are connected to the shielded filter enclosure.

The second terminal of the resistor R44A is connected to the first terminal of a capacitor C39A and also the non-inverting input of an operational amplifier U30A.

In the shielded filter enclosure, in the reference line the second terminal of the resistor R38C is connected to the first terminal of a capacitor C38C and to the first terminal of a resistor R44C. The second terminal of the resistor R38D is connected to the first terminal of a capacitor C38D and to the first terminal of a resistor R44D. The second terminals of the capacitors C38C and C38D are connected to the shielded filter enclosure.

In the shielded amplifier enclosure, in the signal line the second terminal of the resistor R44A is connected to the first terminal of a capacitor C39A. The second terminal of the resistor R44B is connected to the first terminal of a capacitor C39B and also to the first terminal of a resistor R46A. The first terminal of the resistor R46A is also connected to circuit ground. The second terminal of the resistor R46A is connected to the inverting input of the operational amplifier U30A and to the first terminal of a capacitor C40A as well as to the first terminal of a resistor R47A. The second terminal of the capacitor C40A and the second terminal of the resistor R47A are connected to the output of an operational amplifier U40A to provide the signal output S1.

The second terminal of the resistor R44C is connected to the first terminal of a capacitor C39C and to the non-inverting input of an operational amplifier U40A. The second terminal of the resistor R44D is connected to the first terminal of a capacitor C39D and to the first terminal of a resistor R46B as well as to circuit ground. The second terminal of the resistor R46B is connected to the inverting input of the operational amplifier U40A and to the first terminal of a capacitor C40B as well as to one resistive input of a digitally controlled potentiometer U50. The control signals, that is clock, chip select and SD1 are connected to the three digital inputs of the digitally controlled potentiometer U50. The second terminal of the capacitor C40B is connected to the output of the operational amplifier U40A and to the second terminal of the resistor chain of the digitally controlled potentiometer U50. As mentioned above, the second terminals of the capacitors C38A to C38D and C39A to C39D are connected to the shielded amplifier enclosure.

The output of the amplifier U40A is the reference output signal.

The signal appearing on the reference circuit is subtracted from the signal circuit. If impedances and wire pathways are well matched between signal and reference loops, the magnetically induced interference appearing in the signal circuit will be removed by subtraction of the reference signal.

Each resistor designated R32 represents the impedance of body tissue, typically 100 ohms, between signal and ground electrodes. Each resistor designated R39 represents the ground electrode, preferably 10K ohms or less, located typically at the base of the neck. Similarly, each resistance R42 represents the corresponding ground electrode for the associated reference electrodes R41A, R41B etc. Resistors R37 (A through H) represent the resistance of the carbon wire connecting the electrode or reference loop to the electronic amplifiers, combined with the resistance of a patient safety resistor. A typical value for R37 is 13K ohms. The safety resistor typically is 12.5K ohms (range 10K to 15K ohms), preferably non-magnetic (such as Ohmite Macrochip™ SMD resistor), and is mounted in the electrode wire close (within 0.3 m) to the patient.

All of the components associated with the reference mesh and body electrodes may be considered impedances (i.e. having to greater or lesser degrees, resistive, inductive and capacitive components). Thus, except where indicated explicitly to the contrary or where the context does not permit, as used herein, all references to resistance may be regarded as including reference to impedance and “resistive” should be interpreted likewise.

The body electrodes (R30A-etc and R42) are composed of resistive elements at all frequencies and significant capacitive elements down to about 10 Hz. R32, the body tissue beneath the scalp, may be considered to be solely resistive below 100 Hz. R41A-etc in the reference mesh corresponds to R30A-etc, and R40A-etc in the reference mesh corresponds to R32, with the goal being to match these corresponding elements electrically, primarily in the frequency range for physiological signals of interest, 1-1000 Hz. Above that range the electronic filters take over for eliminating magnetic and rf noise. There are capacitive and inductive elements in the reference mesh that are significant at rf, and matching the impedances of the loops at rf is desirable. However, for matching purposes, the maximum tolerable range may be considered to be a DC resistance measured in a reference mesh loop of 50 to 50K ohms (measured at the point where the loop connects to the cable, i.e., in front of resistance R37). A preferred range would be an impedance of between 1K and 10K ohms measured in the reference loop at a frequency of 10 Hz. The body electrode impedances (at 10 Hz) are preferably lower than 10K ohms with a maximum of 20K ohms measured between the signal electrode and ground electrode.

Generally, there may be some level of electrical inter-connection between the points of connection to the reference mesh, depending on the construction. If a continuous conductive fabric or foam is used, there is significant connection throughout the material, and R40A-etc are all connected by primarily resistive and capacitive elements. At the other end of the spectrum, if a lattice network is used, then conductive strings connect the various junctions where R41A-etc. meet R40A-etc. Thus, “reference electrode” is to be interpreted as encompassing the extremes and all possible intermediate forms of construction. The connections are again primarily resistive and capacitive, and can be every junction connected to every other junction at one extreme, or at the other extreme just nearest neighbouring junctions connected.

The nth channel is connected to a neutral location (close to areas of physiological signals of interest but without signal activity) such as behind the ear or on the earlobe for EEG, and has the same configuration (as the signal channels) of a signal loop paired with a matching reference loop. Thus, the n'th channel conveys a compensation signal whilst measurement signals are provided via channels 1 to (n-1). R32 serves as a common ground electrode to the body for all signal circuits, and similarly R42 is a common ground connection to the reference mesh for all the reference circuits. In the nth channel, the amplifiers corresponding to U30A and U30B are designated as U33A and U33B respectively and the digitally controlled potentiometer corresponding to U50 is designated as U60.

The patient cable consisting of all carbon wires twisted in pairs is approximately 2 to 5 meters in length and terminates at the shielded enclosure containing rf filters, analog amplifiers, filters, A/D converters and digital control circuitry. Filtering for rf interference is accomplished with two layers of filters separated by a five-sided shielded enclosure (labelled “Shielded Filter Enclosure” in FIG. 10). The first rf filter begins with resistors R38, 100 to 1K ohms, carbon or thick film composition. Capacitors C38 represent feedthrough capacitors of 1000 pF to 10,000 pF inserted into the wall of the shielded filter enclosure. Alternatively, capacitors C38 may be replaced by a filter connector such as Amphenol™ part number 21-474021-025 which has a pi filter configuration.

Resistors R44 begin the second rf filter (same values and types as R38), with feedthrough capacitors C39 (same values and types as C38) inserted into the wall of the shielded amplifier enclosure. Further rf filtering may be accomplished with the use of a 4-channel common mode choke for the four leads of each channel; and or the addition of a 100 to 1 K ohm resistor followed by a 1 to 5 nF capacitor to ground in the leads to the non-inverting inputs of each preamplifier (pins 3 and 5 of U30 and U40 in FIG. 10), and or the insertion of a 100 to 500 pF capacitor between the inverting and non-inverting inputs of the preamplifiers.

Circuit power ground (common), denoted by the triangle symbol within the shielded amplifier enclosure near the bottom of FIG. 10, is preferably connected to the metallic shield enclosure in one location as shown in the Figure but the shield may also remain isolated from circuit ground. Although circuit power connections are not shown in the Figures, it is understood that the analog integrated circuit amplifiers and filter IC's, etc., are connected to bipolar power supplies of typically ±2.5 volts to ±10 volts, and digital modules are connected to +5 volts. Power is supplied preferably from batteries located within the shielded amplifier enclosure, but may also be supplied from an external power source (isolated medical grade power supply or batteries) if the power inputs are filtered for rf at the shield enclosure, using filters similar to those shown for the signal lines.

The preamplifiers (U30 and U40 in FIG. 10) are typically low noise, high input impedance dual operational amplifiers such as Analog Devices AD8620 or OP2177. On the signal side (U30A and U30B in FIG. 10) a gain of 2 (typical, range 1 to 4) is established by resistors R46 and R47, typically 33K ohms. On the reference side, variable gain is implemented by the use of a digitally controlled potentiometer (U50 and U60 in FIG. 10) in place of R47. This allows the dynamic adjustment of the reference signal gain under programmatic control for maximum interference reduction. Alternatively, R47 on the reference side may be a resistor matched to R47 on the signal side.

High resolution is necessary for precision matching of signal levels in the channels; Analog Devices™ AD7376 with 128 positions, or Analog Devices AD5231 with 1024 steps are examples of digital potentiometers that may be used for U50 and U60. In one example, an AD7376 of 100K ohms is used with R46 and R47 equal to 33K ohms. In this instance, the signal gain is 2 and the reference gain varies from 1 to approximately 4. In another example, an AD5231 of 50K ohms is used with R46 and R47 equal to 17K ohms; In this case the signal gain is again 2, and there reference gain varies from 1 to approximately 4, but the resolution of adjustment is greatly improved with 1024 steps instead of 128. In both cases, the control of the potehtiometer is implemented via three digital control lines, labeled CS, CLK and SDI in FIG. 10. This method of control is desirable as it enables “daisy chaining” the digital potentiometers as shown in FIG. 10, which is advantageous for adjusting reference levels when large numbers of channels are used. Capacitors C40 reduce noise from the digital potentiometers when adjusting; they are used on the signal amplifiers to keep the bandwidths of the signal and reference amplifiers closely matched.

Thus, the overall electrical connection arrangement can be seen more clearly from FIGS. 11 and 12 with signal and reference (with respective ground) electrodes and connections disposed over the scalp of the subject (channels 1-(n-1)).

The nth channel can be seen to comprise the last signal and reference electrodes and connections (with ground electrodes and connections) which are located beneath or on an ear. To repeat, the signal and ground electrodes are in low resistance contact with the skin whilst the reference electrodes (or connections) are part of the mesh which is close to but not in direct (i.e. not in low resistance) contact with the skin.

FIG. 11 shows the conductive pathways for the signal electrodes R30A (scalp electrode) and R30B (ear reference electrode), tracking through the body of the subject and out through the ground electrode R39. In contrast, FIG. 12 shows the conductive pathways for the reference loops associated with scalp and ear reference electrodes. The reference loop electrodes R41A and R41B are connected to a mesh which covers the scalp but is not in direct electrical contact with the scalp. Thus, FIGS. 11 and 12 show the separate circuit loops connected to the amplifying and filtering circuit of FIG. 10.

FIG. 13 shows an installation for embodiments of the invention. A subject and a scanner together with the electronics of, for example, FIGS. 8 to 12, is enclosed inside a scanner room shielded from external interference. The amplifiers and filters of the electronics are connected to the scanner head via the electrode wires and the output signals are converted to optical signals and are transmitted through the walls of the shielded scanner room via fibre optic cables. Outside the shielded scanner room, the fibre optic cables are connected to a fibre optic transceiver where signals are converted back to electrical signals and passed by an Ethernet system to a computer for control purposes, storage, display and printout. The fibre optic system is bidirectional so that the system in the shielded scanner room may be controlled by the computer.

FIG. 14 shows more of the circuitry enclosed in the shielded amplifier enclosure connected to the outputs of the circuitry shown in FIG. 10 for processing the outputs of the circuitry of FIG. 10.

The scalp signal S1 obtained from the output of the amplifier U30A of FIG. 10 is applied to the first terminal of a resistor R50A. The second terminal of the resistor R50A is connected to the first terminal of a resistor R51A and also to the first terminal of a capacitor C50A. The second terminal of the resistor R51A is connected to the first terminal of the capacitor C51A and to the non-inverting input of an amplifier U70A. The second terminal of the capacitor C51A is connected to circuit ground and the second terminal of the capacitor C50A is connected to the inverting input of the operational amplifier U70A and also to the output of the operational amplifier U70A.

Similarly, the reference signal R1 (which is obtained from the output of the operational amplifier U40A in FIG. 10) is applied to the first terminal of a resistor R50B. The second terminal of the resistor R50B is connected to the first terminal of a resistor R51B and to the first terminal of a capacitor C50B. The second terminal of the resistor R51B is connected to the first terminal of a capacitor C51B and to the non-inverting input of an operational amplifier U70B. The second terminal of the capacitor C51B is connected to circuit ground and the second terminal of the capacitor C50B is connected to the inverting input of the operational amplifier U70B and to the output of the operational amplifier U70B.

The output of the operational amplifier U70A is connected to the first terminal of a resistor R52A. The second terminal of the resistor R52A is connected to the non-inverting input of an operational amplifier U71. Similarly, the output of the operational amplifier U70B is connected to a first terminal of a resistor R52B and the second terminal of the resistor R52B is connected to the inverting input of the operational amplifier U71. A capacitor C52A is connected between the inverting and non-inverting inputs of the operational amplifier U71.

The output of the operational amplifier U71 is connected to the first terminal of a resistor R53. The second terminal of the resistor R53 is connected to the first terminal of a resistor R54 and to the gain-setting terminal of the operational amplifier U71. The second terminal of the resistor R54 is connected to circuit ground.

The output of the operational amplifier U71 is also connected to the first terminal of a resistor R55A. The second terminal of the resistor R55A is connected to the first terminal of a capacitor C53A and to the non-inverting input of an operational amplifier U72. The second terminal of the capacitor C53A is connected to a frequency control input of the operational amplifier U72.

Similarly, a ground signal Sn obtained from the output of the amplifier U30B in the circuit of FIG. 10 is applied to the first terminal of a resistor R50C. The second terminal of the resistor R50C is connected to a first terminal of a resistor R51C and to the first terminal of a capacitor C50C. The second terminal of the resistor R51C is connected to a first terminal of a capacitor C51C and to the non-inverting input of an operational amplifier U73A. The second terminal of the capacitor C50C is connected to the inverting input of the operational amplifier U73A and to the output of the operational amplifier U73A.

The corresponding reference signal obtained from the output of the operational amplifier U40B in the circuit of FIG. 10 is connected to the first terminal of a resistor R50D, the second terminal of the resistor R50D being connected to the first terminal of a resistor R51D and to the first terminal of a capacitor C50D. The second terminal of the resistor R51D is connected to the first terminal of a capacitor C51D and to the non-inverting input of an operational amplifier U73B. The second terminal of the capacitor C51D is connected to circuit ground.

The second terminal of the capacitor C50D is connected to the inverting input of the operational amplifier U73B and to the output of the operational amplifier U73B.

The output of the operational amplifier U73A is connected to a first terminal of a resistor R52C and the second terminal of the resistor R52C is connected to the non-inverting input of a further operational amplifier U74. In the reference line, the output of the operational amplifier U73B is connected to a first terminal of a resistor R52D and the second terminal of the resistor R52D is connected to the inverting input of the operational amplifier U74. The capacitor 52B is connected between the inputs of the operational amplifier U74.

The output of the operational amplifier U74 is connected to a first terminal of a variable resistor R56, the second terminal of the variable resistor R56 being connected to the first terminal of a resistor R57 and also to a gain setting input of the amplifier U74. The second terminal of the resistor R57 is connected to circuit ground. The output of the operational amplifier U74 is also connected to the input of a filter integrated circuit U75 which may be set to 50 or 60 Hz.

The centre frequency of the filter U75 is determined by a number of resistors R58, R59, R60 and 61 connected to the appropriate pins of the filter unit U75. The output from the filter unit U75 is connected to the first terminal of a capacitor C60 and to the first terminal of a resistor R62A. The second terminal of the capacitor C60 is connected to the first terminal of a resistor R63 and to the non-inverting input of an operational amplifier U76A. The second terminal of the resistor R62A is connected to the non-inverting input of the operational amplifier U76A and also to the first terminal of a resistor R62B. The second terminal of the resistor R62B is connected to the output of the operational amplifier U76A, to the first terminal of a capacitor C61 and to the first terminal of a resistor R62C. The second terminal of the capacitor C61 is connected to the first terminal of a variable resistor R64 and to the non-inverting input of an operational amplifier U76B. The second terminal of the resistor R62C is connected to the inverting input of the operational amplifier U76B and to the first terminal of a resistor R62D. The second terminal of the resistor R62D is connected to the output of the operational amplifier U76B. The output of the operational amplifier U76B is also connected to the first terminal of a resistor R55B, the second terminal of the resistor R55B being connected to the inverting input of the operational amplifier U72 and to the first terminal of the capacitor C53B. The second terminal of the capacitor C53B is connected to a frequency correction input of the operational amplifier U72.

In FIG. 14, the signal and reference signals are filtered by second order Bessel filters constructed around U70 and U73, which are dual operational amplifiers of the same types as U30 and U40 of FIG. 10. The Bessel filters are low pass, with a cutoff (−3 dB) typically of 145 Hz. Resistors R50 and R51 are 6650 ohms, capacitors C51 are 0.12 μF and capacitors C50 are 0.22 μF for 145 Hz cutoff. The filters must be closely matched in each signal-reference pair to maintain high noise rejection at the differential amplifier; this is achieved by closely matching the filter components preferably to within 0.1% tolerance, or to a maximum of 1% tolerance.

Following the Bessel filters, a differential mode to common mode filter composed of resistors R52 and capacitors C52 (600 ohms and 1.0 μF respectively for a cutoff frequency of 133 Hz) is placed at the input of a wide bandwidth differential amplifier (U71 and U74 in FIG. 14) such as Analog Devices™ AD8129 or similar. The reference loop signal is subtracted at this stage, with an equivalent third order low pass filter of 100 Hz cutoff formed by the combination of filters and differential amplifier. Although low pass filtering is advantageous for minimizing interference, the signal and reference loops must be well-matched in order to minimize interference within the signal bandwidth, 100 Hz in this case.

The gain for the differential amplifier is typically set at 12.5. In FIG. 14, resistors R54 and R53 (221 ohms and 2.55K ohms respectively) set the gain for the signal channels. Channel n, connected to a neutral location on the body near the physiological signals of interest (such as the earlobe or behind the ear for EEG) is used for powerline interference reduction. After rf and magnetically induced interference is filtered and subtracted from channel n, the remaining signal (composed primarily of 50/60 Hz voltages capacitively coupled to the body from the power mains) is subtracted from the EEG signal. Therefore, channel n must be closely matched at 50/60 Hz to the EEG channels, and an adjustable gain control at differential amplifier U74 in FIG. 14 enables matching the gain of channel n to the other channels. The gain range for U74 is set by R57 at 221 ohms, and R56, a 2490 ohms resistor in series with a 100 ohms potentiometer. For maximum powerline rejection, a variable gain control may be added to each EEG channel for individual adjustment, such as replacing R53 with a 2490 ohms resistor in series with a 100′ ohms potentiometer.

Since the signal on channel n is subtracted from the other signal channels, any residual interference appearing on channel n from sources other than 50/60 Hz powerline voltages will appear on the signal channels if it is not matched to the interference on each signal channel. Precise matching of residual interference across channels is not expected, so a means of minimizing any signal other than powerline noise appearing on channel n is necessary.

One method, shown in FIG. 14, is to bandpass filter channel n with a Texas Instruments™ UAF42 filter IC (U75) set at 50 or 60 Hz. For a center frequency of 60 Hz, Q equal to 30, and bandpass gain of 1, R58 is set to 5.49K ohms, R59 and R60 are 834K ohms, and R61 is 487 ohms. Phase adjustment is necessary after filtering to precisely match the phase of the 50/60 Hz signal remaining on channel n to the other signal channels. In FIG. 14, this is implemented with two all pass filter circuits constructed around dual operational amplifier U75 (Texas Instruments TL072 or similar). For 90 degrees of phase shift at 60 Hz, capacitors C60 and C61 are set to 1 μF. Resistor R63 is 265K ohms and resistor R64 is a combination of 261K ohms in series with a 10K ohms potentiometer for phase adjustment. Resistors R62 are 100K ohms. Alternatively, R64 may be replaced with a digitally controlled potentiometer as described above for adjusting amplifier gains, in order to adjust phase shift by programmed means.

An alternative approach (not shown) is to use a bandpass filter with lower Q to allow a passband of 50 to 60 Hz, and follow with a phase locked loop to lock onto the powerline noise. The output of the phase locked loop is phase adjusted and the gain may be trimmed to match the powerline interference appearing on the signal channels. The filtered and phase adjusted powerline interference signal on channel n is subtracted from the signal channels using a differential amplifier (U72 in FIG. 14, Analog Devices AD620 or similar). Resistors R55 (1K ohms) and capacitors C51 (150 pF) filter high frequency noise appearing at the output of the wide bandwidth differential amplifier U71, and match the inputs at U71.

In FIG. 15, the main stages of signal amplification and additional filtering are shown.

Signal S1 obtained from the output of the operational amplifier U72 in the circuit of FIG. 14 is applied to the first terminals of further resistors R70A and R71A as shown in FIG. 15. The second terminal of the resistor R70A is connected to the first terminal of a capacitor C70A and to the non-inverting input of a further operational amplifier U80. The second terminal of the resistor R71A is connected to the first terminal of a capacitor C71A and to the inverting input of the operational amplifier U80. The second terminals of the capacitors C70A and C71A are taken to circuit ground. The output of the operational amplifier U80 is taken to a first terminal of a resistor R72A and the second terminal of the resistor R72A is connected to the first terminal of a resistor R73B and to the first terminal of a resistor R74B as well as to the input of a filter U81. The second terminals of the resistors R73B and R74B are taken to the filter control terminals of the filter U81. The output of the filter U81 is connected to a first terminal of a resistor R75A and the second terminal of the resistor R75A is connected to the first terminals of resistors R76A and R77A. The second terminal of the resistor R77A is taken to a filter control terminal of the filter U81. The second terminal of the resistor R76A is connected to a filter control terminal of the filter U81. The output of the filter U81 is connected to a second terminal of a resistor R75A and to the first terminal of a resistor R78C as well as to a first terminal of a resistor R78D. The second terminal of resistor R78C is connected to the non-inverting input of an operational amplifier U82. The second terminal of the resistor R78D is connected to the first terminal of a capacitor C72C and to the inverting input of the operational amplifier U82. The second terminal of the capacitor C72C is taken to circuit ground. The output signal S1 with reduced interference is obtained from the output of the operational amplifier U82.

The ground signal Sn taken from the output of the operational amplifier U74 in the circuit of FIG. 14 is connected, as shown in the circuit of FIG. 15, to the first terminal of a resistor R90 and to the first terminal of a capacitor C90. The second terminal of the resistor R90 is connected to the first terminal of resistor R91 as well as to the first terminal of a capacitor C92. The second terminal of capacitor C90 is connected to the first terminal of a resistor R92 and to the first terminal of a capacitor C93. The second terminal of the capacitor C92 is connected to the second terminal of the resistor R92. The second terminal of the resistor R91 is connected to the non-inverting input of an operational amplifier U83A. The inverting input of the operational amplifier U83A is connected to the output of the operational amplifier U83A.

The second terminal of the capacitor C93 is connected to the inverting input of a further operational amplifier U83B and to the output of the operational amplifier U83B. The non-inverting input of the operational amplifier U83B is connected to the slider of a variable resistor R95. The first terminal of the resistor R95 is connected to the output of the operational amplifier U83A and the second terminal of the resistor R95 is connected to circuit ground.

The output of the operational amplifier U83A is further connected to the first terminals of two resistors R96B and R97B. The second terminal of the resistor R96B is connected to the first terminal of a capacitor C94B and to the non-inverting input of a further operational amplifier U84. The second terminal of the resistor R97B is connected to the first terminal of a capacitor C95B and to the inverting input of the operational amplifier U84. The second terminals of the capacitors C94B and C95B are connected to circuit ground.

The output of the operational amplifier U84 is connected to the first terminal of a resistor R98B. The second terminal of the resistor R98B is connected to the input of a filter unit U85 and to the first terminals of two resistors R99B and R100B. The second terminals of the resistors R99B and R100B are connected to the filter control terminals of the filter unit U85.

The second terminal of the resistor R100B is connected to the first terminal of a resistor R101B and the second terminal of the resistor R101B is connected to a filter control terminal of the filter unit U85 and to the first terminals of two resistors R102B and R103. The second terminal of the resistor R102B is connected to the filter control terminal of the filter unit U85 and the output of the filter unit U85 is connected to a second terminal of the resistor R103B and to the first terminals of two resistors R104 and R105. The second terminal of the resistor R104 is taken to the non-inverting input of an operational amplifier U86 and the second terminal of the resistor R105 is taken to the first terminal of a capacitor C96 and to the inverting input of the operational amplifier U86. The second terminal of the capacitor C96B is taken to circuit ground. The ear reference signal with the 50/60 Hz interference removed is obtainable from the output of the operational amplifier U86.

At the input to U80 (differential amplifier such as Analog Devices™ AD627), the signal channel is high pass filtered to remove DC offsets appearing at the electrode interface to the body. Typical values for components are: R70, 39.2K ohms, R71, 1.6M ohms, C60, 0.01 μF, and C61 0.1 μF. Gain for this stage is set at 10. Following is a fourth order Butterworth low pass filter with a cutoff frequency of 256 Hz. This may be implemented using a Linear Devices™ LTC1563-2 filter (U81 in FIG. 15) with resistors R72 through R77 set to 10M ohms. Additional gain of 50 and DC offset filtering is added at U82 and U86 (AD627 typically) with R71, R78, R97, R104 and R105 set to 1.6M ohms and C71, C72, C95, and C96 at 0.1 μF.

Although all channels have the same amplification and filtering as outlined above, channel n has an additional filter as shown in FIG. 15. Since channel n is the ear reference channel, the primary signal appearing on this channel is a large 50/60 Hz signal. As previously described, this signal is subtracted from the signal channels to remove powerline interference. However, in some applications, it may be necessary to observe channel n in order to adjust the reference loop gain for minimizing rf and magnetically induced interference. Therefore, the original channel n signal appearing at the output of U74 in FIG. 14 is routed through a 50 or 60 Hz notch filter in FIG. 15 before amplification and digitization for display. A 60 Hz notch filter is built around operational amplifier U83 (Texas Instruments™ TL072 or similar) using component values shown in FIG. 15, resulting in approximately 45 dB of rejection at 60 Hz, sufficient for displaying channel n without excess powerline noise swamping the trace.

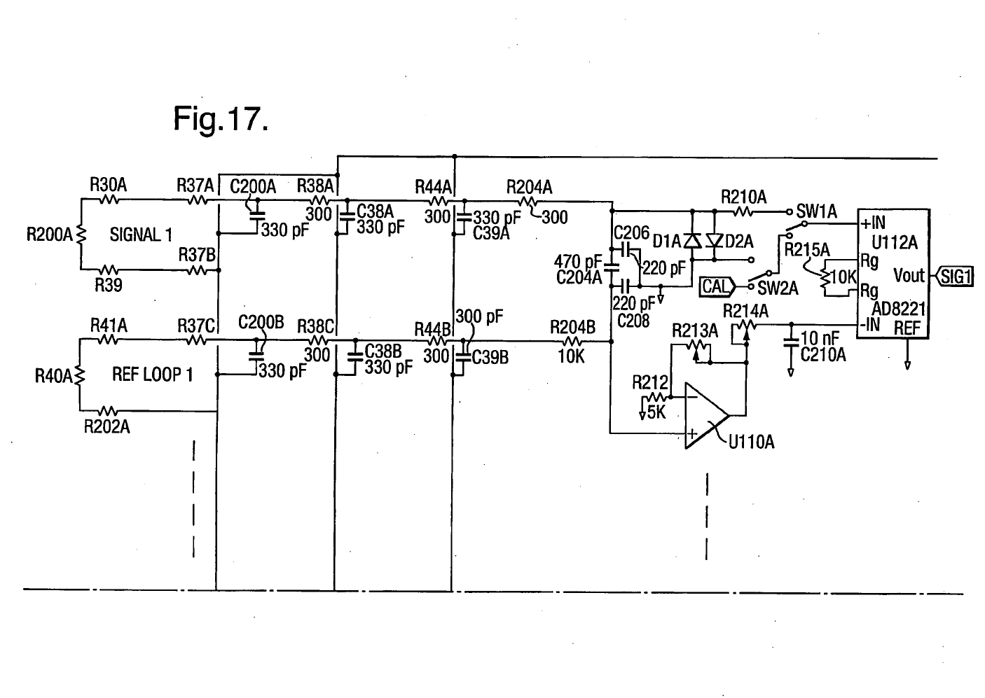

In FIG. 16, the final components of the system are shown.

The noise reducing apparatus is mounted in a shielded amplifier enclosure 1000. The channel signals S1 to Sn-1 from each channel output from the apparatus of FIG. 15 are taken from the outputs of U82 (for the channels 1 to n-1) and from the amplifier U86 for channel n, to the input of a sample and hold unit U100. The sample signals output from the hold unit U100 are applied to the input of a gain analogue-to-digital conversion and multiplexing unit 1001 and the digital outputs from the unit 1001 are applied to the inputs of a central processing unit 1002. The outputs from the central processing unit 1002 are in Ethernet form and are applied to a fibre optic transceiver 1003. Two fibre optic links 1004, 1005 (one for transmission and one for reception) pass through the walls of the shielded amplifier enclosure 1000 and a shielded scanner room 1006. In an exterior control room 1007, the fibre optic cables 1004 and 1005 are connected to the inputs of a further fibre optic transceiver 1008. The Ethernet output from the transceiver 1008 may be connected to a computer 1009 (such as a laptop or a PC) and/or to the internet 1010. A control signal is passed from the unit 1001 back to the unit U100.

U100 represents sample and hold amplifiers for each channel, enabling simultaneous sampling for all channels to avoid distortion of signal samples due to time skewing. After further optional gain adjustment, the sampled signals are digitized to 16 bit resolution. A commercially available 32 channel analog I/O module such as Diamond Systems™ Diamond-MM-32-AT on a PC/104 bus may be used for analog to digital conversion. Further digital control is performed using a CPU such as a Diamond Systems Promethius™ PC/104 CPU module. Software for controlling timing of sampling, digitization, communication over ethernet and other functions is loaded into the PC/104 CPU module.

Communication with the external world is accomplished via, an Ethernet connection, the fiber optic link is inserted between the PC/104 CPU and the network connection outside the shielded MRI scanner room to avoid conducting interference into the shielded room on metallic wires. The fiber optic link also minimizes rf interference leaking into or out of the shielded amplifier enclosure, and for patient safety isolates the amplifier electronics from AC power leakage through the network connection. Fiber optic conversion may be accomplished using a Telebye Model 373 10 Base-T (ethernet) to Fiber Optic Transceiver. Communication with the PC/104 CPU via networking enables command of the system from remote locations (such as the MRI control room) and allows data to be delivered to multiple locations for recording, display and analysis (anywhere on the internet, essentially). Commands from the external computer control initiate functions of the PC/104 CPU, including sampling, reference gain adjust, real time data display, data dump for permanent recording, etc. Although data is temporarily stored in the PC/104 CPU, it is transferred to data storage such as a computer hard drive for permanent recording.

In the embodiments of FIGS. 8-16, signal and reference lines are in close physical proximity along substantial parts of their mutual lengths. Reference signals on the reference lines are at least partly subtracted from the respective measurement signals on their associated measurement signal lines to help reduce interference.

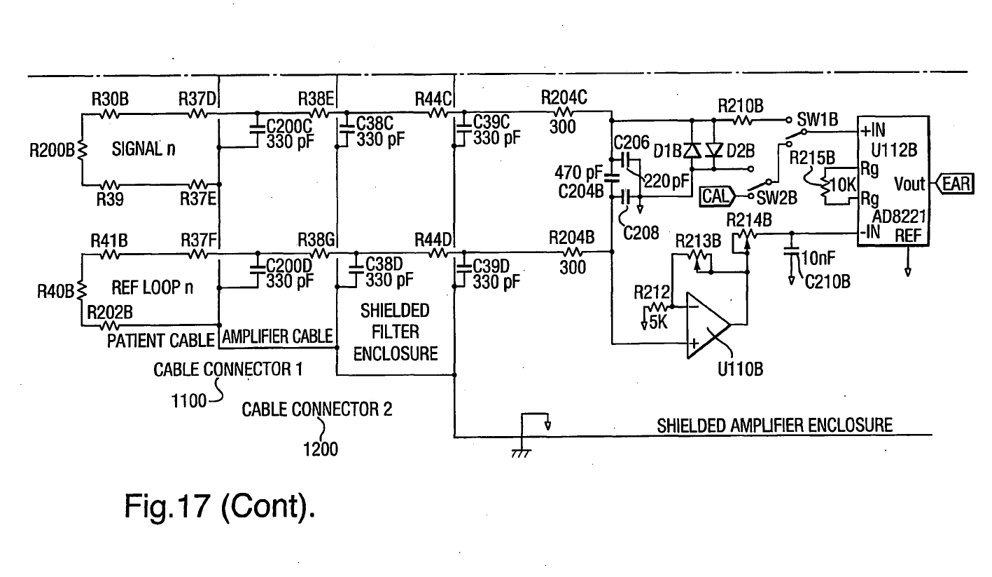

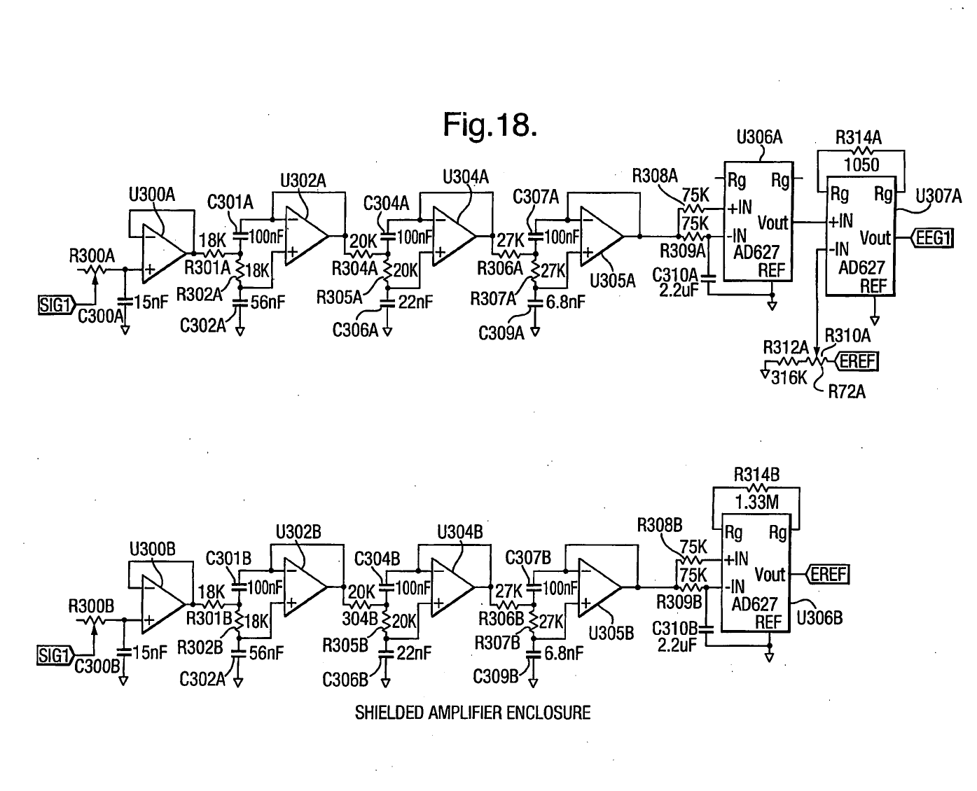

FIGS. 17 and 18 show an embodiment which is an example of a class of particularly preferred embodiments. These embodiments employ one or more measurement channels each comprising a measurement signal line and a reference signal line. The measurement and reference signal lines are twisted together along most of their mutual lengths each having an associated ground line, also closely physically associated therewith.

The components designated R30A etc to R46A etc and C39A etc, for signal 1/reference 1 to signal n/reference n have the same meanings or functions as shown in FIG. 10 and their values are the same as for FIG. 10 except where stated to the contrary. As described further hereinbelow, in the signal processing circuitry, reference line signals are subtracted from their corresponding measurement signals.

As also with the embodiment of FIG. 10, the n'th signal electrode is connected to the patient's skin at a neutral location such as behind the ear or on the earlobe and the corresponding n'th reference electrode is connected to a point on the reference mesh/cap, close to the n'th signal electrode. Thus, the wiring to signal electrodes 1 to (n-1) convey measurement signals and the wiring to the n'th signal electrode provides a compensation signal. As will be further described hereinbelow, the compensation signal may be used to derive interference components used separately to reduce interference on each measurement signal.

Referring specifically now to FIG. 17, the first and last channels of a system with n channels are shown, with n ranging from 2 to 1024.

The electrodes and reference sources are coupled to the patient subject cable connected to the amplifier cable via a cable connector 1100. The amplifier cable is connected via a cable connector 1200 to the shielded filter enclosure which is mounted on the shielded amplifier enclosure. In the patient cable, R30A (as in FIG. 10) represents the electrode impedance. A first terminal of the resistor R30A is coupled to a resistor R200A representing the impedance of the body tissue, and the second terminal of the resistor R30A is connected to a first terminal of the resistor R37A which represents the impedance of the conductor connecting the signal electrode to the cable connector 1100 in the patient cable. The second terminal of the resistor R200A is connected to a first terminal of the resistor R39 which represents the impedance of the circuit ground electrode. The second terminal of the resistor R39 is connected to the resistor R37B which represents the impedance of the conductor connecting the circuit ground electrode to the cable connector 1100.

The resistor R41A represents the impedance of the connection of the conductor to the reference mesh. A first terminal of the resistor R41A is connected to the resistor R40A representing the impedance of the reference mesh (as in FIG. 10) and the second terminal of the resistor R41A is connected to the resistor R37C representing the impedance of the conductor. The second terminal of the resistor R37C is connected via the cable connector 1100 to the amplifier cable. The second terminal of the resistor R40A is connected to a first terminal of a resistor R202A which represents the impedance of the connection from the reference mesh to the ground conductor. The second terminal of the resistor R202A is connected at cable connector 1100 to circuit ground.

In the amplifier cable, the second terminal of the resistor R37A is connected via cable connector 1100 to a first terminal of a capacitor C200A and to the first terminal of the resistor R38A. The second terminal of the capacitor C200A is connected to circuit ground. The second terminal of the resistor R38A is connected via cable connector 1200 into the shielded filter enclosure. The second terminal of the resistor R37C in the patient subject cable is connected via the cable connector 1100 to the first terminal of the capacitor C200B and also to the first terminal of the resistor R38C. The second terminal of the capacitor C200B is connected to circuit ground and the second terminal of the resistor R38C is connected via cable connector 1200 into the shielded filter enclosure.

In the shielded filter enclosure, the second terminal of the resistor R38A is connected to the first terminal of the capacitor C38A and to the first terminal of the resistor R44A (as in FIG. 10). The second terminal of the capacitor C38A is connected to circuit ground. The second terminal of the resistor R44A is connected in the shielded amplifier enclosure to a first terminal of capacitor C39A and to the first terminal of a resistor R204A. The second terminal of the capacitor C39A is connected to circuit ground.

In the shielded filter enclosure, the second terminal of the resistor R38C is connected to a first terminal of capacitor C38B and to the first terminal of the resistor R44B. The second terminal of the capacitor C38B is connected to circuit ground.

In the shielded amplifier enclosure, the second terminal of the resistor R44B is connected to a first terminal of the capacitor C39B and to the first terminal of a resistor R204B. The second terminal of the capacitor C39B is connected to circuit ground.

The second terminal of the resistor R204A is connected to a first terminal of a capacitor C204A, to the first terminal of another capacitor C206, to the cathode of a diode D1A, to the anode of a further diode D2A and to the first terminal of a resistor R210A.

The second terminal of the resistor R204B is connected to the second terminal of the capacitor C204A, to the first terminal of a further capacitor C208 and to the non-inverting input of an operational amplifier U110A. The second terminals of the capacitors C206 and C208 are connected to circuit ground. The anode of the diode D1A is connected to circuit ground and the cathode of the diode D2A is also connected to circuit ground. The inverting input of the amplifier U110A is connected to a first terminal of a resistor R212 and the first terminal of a variable resistor R213A. The second terminal of the resistor R212 is connected to circuit ground.

The second terminal of the variable resistor R213A is connected to the output of the amplifier U110A. The output of the amplifier U110A is also connected to a first terminal of a variable resistor R214A and the second terminal of the resistor R214A is connected to a first terminal of a further capacitor C210A and to the inverting input of an instrumentation amplifier U12A. The second terminal of the capacitor C210A is connected to circuit ground.

The second terminal of the resistor R210A is connected to a first contact of a switch SW1A. The second contact of the switch SW1A is connected to the wiper of a further switch SW2A. The wiper of the switch SW1A is connected to the non-inverting input of the instrumentation amplifier U112A. A first contact on the switch SW2A is connected to circuit ground and the second contact of the switch SW2A is connected to a calibration terminal. A gain setting resistor R215A is connected to the gain setting terminals on the instrumentation amplifier U112A. Circuit ground is connected to the shielded amplifier enclosure.

The above components comprise a first channel.

The system of FIG. 17 shows a plurality of n channels, the second to the nth channels preferably being identical to the first channel described above. The first to the n-1 channels are connected to the electrodes on the scalp of a subject and the nth channel is connected to a neutral location such as an ear lobe. For the second to the nth channels, the corresponding reference numerals have been denoted by the same numerical reference but with different alphabetical references.

Channel 1 is a signal channel typically connected to the scalp for EEG by means of an electrode with an electrical impedance represented by resistor R30A, preferably 5000 ohms or less at 10 Hz. All electrodes are constructed of a resistive material such as carbon-loaded plastic, press-molded carbon powder, or the bare ends of carbon wire. Electrical contact between electrode and body is facilitated by a conductive paste of the type commonly used for electrophysiological measurements. R200A represents the impedance of body tissue, about 100 ohms. R39 represents the circuit ground electrode to the body, preferably of 5000 ohms impedance or less at 10 Hz, located typically at the base of the neck. R37A represents the combined resistance of the carbon wire connected to the electrode and the resistance of a patient safety resistor. A typical value for R37A is 13K ohms. The safety resistor typically is 12.5K ohms (range 10K to 15K ohms), preferably non-magnetic (such as Ohmite Macrochip SMD resistor), and is mounted in the patient cable side of cable connector 1100 in FIG. 17 close (within 0.3 meters) to the patient. Similarly, R37B is the combined resistance of the carbon wire connected to the ground electrode and a patient safety resistor.

For each signal electrode, the companion ground wire is twisted tightly with the electrode wire to minimize the loop area formed by the wires and hence minimize induced magnetic field interference in the signal. Capacitor C200A, typically 330 pF, is located in the amplifier cable side of cable connector 1100 and acts in combination with R37A to filter radio frequency (rf) interference appearing in the signal line. The ground wire is connected via R37B to the shield of the amplifier cable, which is connected to isolated circuit ground at the shielded amplifier enclosure. Similarly, R30B, R200B, R37D, R37E and C200C represent components of signal channel n.

R41A represents the resistance of the connection of a carbon or copper wire to a conductive reference mesh that spans the surface of the head but is not in electrical contact with the body. The purpose of the reference mesh is to allow the formation of a reference loop (labeled “Ref Loop 1” in FIG. 17) spatially matching and electrically isolated (except for a common circuit ground) from the loop formed by the electrode and ground wires (labeled “Signal 1” in FIG. 1). Since the voltage on the reference loop arises primarily from magnetically induced interference, subtracting it from the voltage in the signal channel results in the removal of magnetically induced interference in the signal. R41A must be located spatially very close to R30A to closely match the signal and reference loops. R40A represents the resistance of the reference mesh. R202A is the resistance of the connection from the mesh to the ground wire, and must be located spatially very close to R39. The wires for the reference loop are twisted together tightly to minimize loop area, and the pair is twisted together with the electrode wire pair to closely match the paths followed by the reference and signal wires for the channel. R37C represents a resistor of 300 to 15K ohms located in the patient cable side of cable connector 1100, acting in combination with capacitor C200B, typically 330 pF, located in the amplifier cable side of cable connector 1100, for the purpose of filtering rf interference appearing in the reference loop. The ground wire for the reference loop is connected directly to the shield of the amplifier cable. Similarly, R41B, R40B, R202B, R37F and C200D represent components of reference loop n for reducing interference in signal channel n.

The resistances in the reference loops (R41, R40 and R202) are low in value (preferably less than 500 ohms each and not more than 1000 ohms total sum for each loop) to minimize the level of electrostatic interference induced in the reference loop from external sources. Compensation for the difference signal in signal electrode impedance versus reference loop resistance is implemented in the amplifier front end circuitry, as described below. Carbon wire may be used for connecting to the reference mesh if resistivity is kept low, but copper wire is preferable. The reference mesh is constructed with flexible, conductive fabric, preferably with elastic properties to provide a snug fit on the head. One example of an acceptable material is “See-Through Conductive Fabric”, # N208 (supplied by Less EMF, Inc., Albany, N.Y.), which is a nylon knit fabric with a silver coating yielding less than 5 ohm/square electrical resistivity. Typically the reference mesh is attached to an electrode cap by stitching, or hook and loop, with small holes cut at the appropriate locations in the reference mesh to allow clearance for scalp electrodes. The electrode cap serves to hold scalp electrodes in place and electrically insulate the reference mesh from the body. Reference loop wires may be attached to the reference mesh by mechanical means such as inserting the wire through the weave of the reference mesh and stitching in place, or a small hook and loop, or bonding in place by means of a conductive epoxy. A second layer of electrical insulation may be placed over the top of the reference mesh and its wire connections, either with insulating fabric or by coating the reference mesh with an insulting material such as a thin layer of latex rubber. Alternatively, the reference mesh may also double as an electrode cap if an electrically insulating coating or barrier is added to both sides of the reference mesh.

There are allowable variations in the arrangement of the ground wires. One acceptable configuration consists of each signal wire paired with a ground wire tightly twisted for the complete path followed from the body to the amplifier. In this case the reference loop has a similar configuration, with a corresponding ground wire tightly twisted for the complete path followed from reference mesh to amplifier. With this configuration, each channel has four wires, and the ground wires terminate at the chassis of the shielded filter or amplifier enclosure. A variation of this approach has the ground wires terminating on the shield of the amplifier cable as described above. A second type of wiring configuration eliminates the ground wires for each channel in lieu of a single ground wire for all the signal channels, and a single ground wire for all the reference loops. In this case, the patient safety resistors for the ground wires (R37B and R37E in FIG. 17) are reduced to a single safety resistor connected to a single ground wire coming from ground electrode R39. Similarly, the reference loop ground connections (R202A and R202B in FIG. 17) are reduced to a single connection and single ground wire. With this configuration, each channel has two wires, signal and reference loop, tightly twisted together, and there is a single pair of ground wires, also tightly twisted together. The ground wires may terminate at the shield of the amplifier cable, or the chassis of the shielded filter or amplifier enclosure as described previously. Yet another variation uses only a single ground line for both the signal and reference loops. In that case, the reference loop ground connections (R202A and R202B in FIG. 17) terminate at the patient ground electrode R39.

The lower channel in FIG. 17 (nth channel) is connected to a neutral location with respect to physiological signals of interest (such as behind the ear or on the earlobe for EEG), and has the same configuration as the signal channels, consisting of a signal loop paired with a matching reference loop. This channel is used to reduce electrostatic and ballistocardiogram (BCG) interference as will be seen.

The patient cable consisting of all signal, reference loop and ground wires may extend approximately 2 to 5 meters (and preferably approximately 2.5 to 5 meters) in length from the body and terminate at the shielded filter or amplifier enclosure containing rf filters, analog amplifiers, filters, A/D converters and digital control circuitry. In this case, the patient safety resistors must be inserted in the electrode wires within approximately 0.3 meters of the body. Alternatively and preferably, the patient cable extends a short distance from the body (approximately 0.3 meters) and terminates in a multi-conductor connector (located at cable connector 1100 in FIG. 17) for mating with an amplifier cable extending from the amplifier enclosure. As shown in FIG. 17 and described above, rf filtering may be incorporated with patient safety in the mating halves of cable connector 1100. The amplifier cable consisting of multiple twisted pairs of copper wire within a shield extends 2.5 to 5 meters from cable connector 1100 to cable connector 1200, located at the shielded filter enclosure as shown in FIG. 17.

Alternatively cable connector 1200 may terminate at the shielded amplifier enclosure if the additional rf filtering afforded by the use of a shielded filter enclosure is not required. Another alternative dispenses with cable connector 1200 and has the amplifier cable permanently attached to either the shielded filter enclosure or the shielded amplifier enclosure. In the preferred case, as shown in FIG. 17, a cable connector is used, with a first rf filter comprised of resistors R38A etc, typically 300 ohms but ranging in value from 100 to 1000 ohms, of carbon or thick film composition, located in the cable connector on the amplifier cable. Capacitors C38A etc, typically 330 pF but ranging in value from 100 to 1000 pF, are incorporated within the housing of the mating cable connector mounted on the wall of the shielded filter enclosure. Alternatively, if the cable is permanently attached, capacitors C38 are feedthrough types mounted in the wall of the shielded filter enclosure. Resistors R44 begin the second rf filter (same values and types as R38), with feedthrough capacitors C39 (same value range as C38) inserted into the wall of the shielded amplifier enclosure. Further rf filtering may be accomplished with the use of a 2-channel common mode choke inserted in the signal and reference lines of each channel, or the addition of a 100 to 1000 ohm resistor followed by a 200 to 500 pF X2Y capacitor C204A to ground across the input pairs of each channel as shown in FIG. 17.

Circuit power ground, or common rail, denoted by the triangle symbol within the shielded amplifier enclosure near the bottom of FIG. 17, is connected to the metallic shielded enclosure in one location as shown in the Figure. Although circuit power connections are not shown in the Figures, it is understood that the analog integrated circuit amplifiers and filter IC's 18-21, etc., are connected to bipolar power supplies of typically +−2.5 volts to +−10 volts, and digital modules are connected to +5 volts. Power is supplied preferably from batteries located within the shielded amplifier enclosure, but may also be supplied from an external power source (isolated medical grade power supply or batteries) if the power inputs are filtered for rf at the shield enclosure, using filters similar to those shown for the signal lines.

For purposes of patient safety, diodes D1 and D2 shown in FIG. 17 are placed in reverse polarity configuration to circuit ground on every signal line extending from an electrode connected to the body. The diodes are common signal diodes with a forward voltage of approximately 0.6 volts, working in combination with the patient safety resistors to limit leakage currents to the body in the case of a fault in the amplifier circuitry. Resistors R210, typically 1000 ohms, limit current flow in the diodes. Switches SW1 and SW2 in FIG. 17 enable channel selection, injection of a calibration signal (“CAL” source in FIG. 17) and electrode contact impedance testing operations. The switches typically are solid state analog switches such as MAX393 (Maxim Integrated Products, Sunnyvale, Calif.) with low leakage current, and are digitally controlled by software command.

The subtraction of magnetically induced interference in each channel is accomplished with the use of instrumentation amplifiers U112 in FIG. 17, exhibiting high common mode rejection (typically 100 dB or better) and low noise in an extended bandwidth. An example of this type of device is the AD8221, manufactured by Analog Devices, Norwood, Mass. The instrumentation amplifier is also required to have extremely high input impedance, making it suitable for connection to signal sources with high impedance such as electrophysiological electrodes, thus eliminating the need for impedance-matching preamplifiers on the signal input. On the reference loop input, however, variable amplitude and phase adjustment of the magnetically induced interference present in the reference loop is used to compensate for the difference in signal and reference impedances, thus achieving maximum noise rejection in the subtraction process.

Amplifiers U110 and associated circuitry in FIG. 17 constitute a preferable means for enabling the adjustment. U110 is a low noise operational amplifier such as the OP1177 manufactured by Analog Devices, Inc. Digitally controlled potentiometers may be used for R213 and R214 to enable dynamic adjustment under software control or pre-adjustment based on calibration values for a particular electrode cap. A single Analog Devices AD5231 dual channel digital potentiometer with 1024 steps of adjustment and nominal value of 20K ohms may be used for both controls of each channel. The control of the potentiometers is implemented via three digital control lines in a “daisy chain” configuration which is advantageous for adjusting large numbers of channels. The gain of the instrumentation amplifiers U112 is typically set at approximately 6 using resistors R215, and matched across channels using 0.05% tolerance resistors.

In FIG. 18, one signal channel and the ear channel are shown, but it is understood that multiple signal channels in addition to those shown are contemplated, similar to FIG. 17.

FIG. 18 shows the filtering section of the apparatus according to an embodiment of the invention. A signal from the output of the instrumentation amplifier U112A in FIG. 17 is connected to a first terminal of a variable resistor R300A. The second terminal of the variable resistor R300A is connected to a first terminal of a capacitor C300A and to the non-inverting input of an operational amplifier U300A. The second terminal of the capacitor C300A is connected to circuit ground and the inverting input of the operational amplifier U300A is connected to the output of the operational amplifier U300A. The output of the operational amplifier U300A is also connected to a first terminal of a further resistor R301A and second terminal of the resistor R301A is connected to a first terminal of a capacitor C301A and to a first terminal of a resistor R302A. The second terminal of the capacitor C301A is connected to the inverting input of a further operational amplifier U302A and to the output of the amplifier U302A. The second terminal of the resistor R302A is connected to the non-inverting input of the amplifier U302A and to the first terminal of a capacitor C302A. The second terminal of the capacitor C302A is connected to circuit ground.

The output of the amplifier U302A is connected to a first terminal of a resistor R304A. The second terminal of the resistor R304A is connected to the first terminal of a capacitor C304A and to the first terminal of a resistor R305A. The second terminal of the capacitor C304A is connected to the inverting input of an amplifier U304A and to the output of the amplifier U304A. The second terminal of the resistor R305A is connected to the non-inverting input of the amplifier U304A and to the first terminal of a capacitor C306A. The second terminal of the capacitor C306A is connected to circuit ground. The output of the operational amplifier U304A is further connected to a first terminal of a resistor R306A. The second terminal of the resistor R306A is connected to the first terminal of a capacitor C307A and to the first terminal of a resistor R307A. The second terminal of the capacitor C307A is connected to the inverting input of an operational amplifier U305A and to the output of the amplifier U305A. The second terminal of the resistor R307A is connected to the non-inverting input of the amplifier U305A and to the first terminal of a capacitor C309A. The second terminal of the capacitor C309A is connected to circuit ground.

The output of the amplifier U305A is connected to the first terminal of a resistor R308A and the first terminal of a resistor R309A. The second terminal of resistor R308A is connected to the non-inverting input of an amplifier U306A. The second terminal of the resistor R309A is connected to the inverting input of the amplifier U306A and to the first terminal of a capacitor C310A. The second terminal of the capacitor C310A is connected to circuit ground.

The output of the amplifier U306A is connected to the non-inverting input of a further amplifier U307A. The inverting input of the amplifier U307A is connected to the slider of a resistor R310A. The first terminal of R310A is connected to the reference voltage (E_{ref}) and the second terminal of the resistor R310A is connected to the first terminal of resistor R312A. The second terminal of resistor R312A is connected to circuit ground.

A resistor R314A is connected between the gain setting terminals of the amplifier U307A.

The above description relates to a first channel and the second to the n-1th channels are identical to the channels described above. For the nth channel (the ear channel) this circuit is identical up to the amplifier U306A described above except that a gain setting resistor R314B is connected between the gain setting terminals of the corresponding amplifier U306B and the amplifier U307A is omitted.

All channels are filtered by 7^{th} order low pass filters implemented with operational amplifiers U300 through U305 and associated components. U300 through U305 may be implemented in a single integrated circuit, low noise, low offset quad op amp package such as the Analog Devices OP4177. The type of low pass filter used may range from Bessel to Butterworth. The Bessel filter has better step response (less overshoot and ringing) than the Butterworth, but the Butterworth has better rejection of noise than the Bessel. In this example, a compromise filter known as 0.05° Equiripple filter having characteristics midway between Bessel and Butterworth is used to minimize filter ringing but maintain acceptable noise rejection. All of the resistors (R301 through R306) in the filters are 0.05% tolerance, and the capacitors are 2% tolerance. A phase adjustment for each channel is implemented with variable resistor R300, which may be a digitally controlled potentiometer such as the AD5231. This adjustment allows precision phase matching of each channel to the ear channel for the purpose of electrostatic noise rejection, particularly for AC powerline sources.

DC electrode offset potentials are removed from each channel by the use of instrumentation amplifiers U306 (Analog Devices AD627 or similar) and associated components as shown in FIG. 18. In addition, the signal is amplified by a factor of five in this stage in the signal channels. In the ear channel, the signal is amplified by a slightly higher gain, set by resistor R314B. The output of the ear channel, labeled “EREF” in FIG. 18 is then fed into the inverting input of the final stage instrumentation amplifier for each signal channel (U307 in FIG. 18, AD627 or similar), for the purpose of subtracting interference from electrostatic sources such as AC powerline and fMRI appearing on the body and in the signal leads. Additionally, BCG in the signal channels is reduced with this method. In order to closely match the interference appearing on EREF to the interference appearing in each signal channel, a voltage divider consisting of resistors R310A and R312A in FIG. 18 is used to adjust the amplitude of EREF for each signal channel. R310A may be a digitally controlled potentiometer, preferably one channel of a dual channel AD5231 of 20K ohms nominal resistance, with the other channel implemented as R300A for the phase adjustment in the channel. With this configuration, a single integrated circuit controls the amplitude and phase adjustment for electrostatic and BCG interference reduction in each channel. The AD5231 integrated circuits can be daisy-chained with the AD5231 integrated circuits used for magnetic interference reduction as described previously. Resistors R314 set the gain of amplifiers U306 and U307 to 200.

The overall configuration of the system is exactly as shown for the embodiment of FIG. 16. Connection of the scanner and associated components to the outside world is exactly the same as described in respect of FIG. 13.

In addition to the essential amplification and filtering circuitry as described above, it is also contemplated that amplified reference loop signals may be required for software filtering operations. In this case, an individual reference loop is amplified by a factor of 2 to 10, and optionally filtered using the same low pass filter as used for the signal channels (such as the 7 pole 0.05 deg. Equiripple low pass filter shown in FIG. 18). Further gain may be required post filter, up to a factor of 1000. The reference loop signals are subsequently sampled and digitized simultaneously with the signal channel outputs as described previously.

In yet another embodiment of the invention, individual reference loops for each signal channel are replaced by regional reference loops that serve to reduce interference in groups of signal channels. For example, a reference loop may be implemented as described previously for a scalp electrode. This same reference loop may then be used as the reference input for the four surrounding scalp electrodes. Although the match in interference between signal and reference loops may not be as precise for the neighbouring electrodes as the centre electrode, adjustment of the gain and phase of the reference input as shown in the previous example for each of the neighboring electrodes will result in improved noise rejection. An extreme example of this approach is the use of one single reference loop for all signal channels. In this case the gain and phase of the reference loop requires a large range of adjustment across all channels, and may result in less noise rejection than is the case for individual reference loops for each electrode or small neighborhoods of channels.

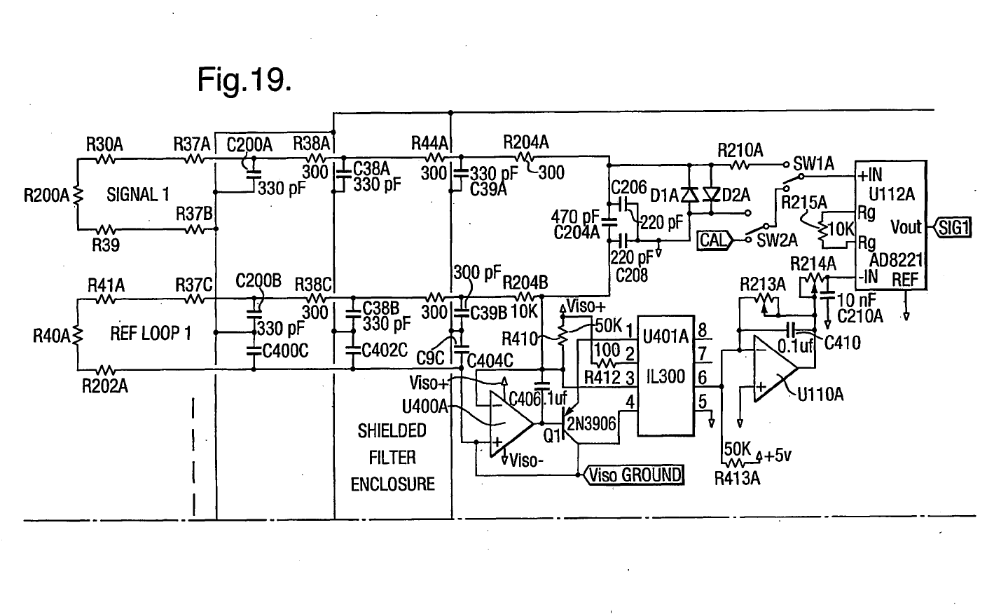

In still yet another embodiment of the invention, the reference loop grounds are electrically isolated from the measurement signal grounds prior to the subtraction stage. This has the effect of reducing magnetically induced interference voltages arising in the loop formed between the signal and reference loops when a common ground is used for both. An example of the isolated type of embodiment is shown in FIG. 19, which is the embodiment of FIG. 17 with the addition of electrical isolation between the signal loop ground and reference loop ground. In this case, the reference loop ground connections (as designated by resistors R202A and R202B) are not connected to the amplifier power supply ground (via the shielded amplifier enclosure as previous), but instead to an isolated ground designated “Viso GROUND” of a separate bipolar power supply, designated “Viso+” and “Viso−”. The isolated power supply may be obtained by batteries, or externally by means of a medically approved isolated bipolar power supply with appropriate rf filtering on the supply leads entering the shielded amplifier enclosure. In this example, the isolated power supply is + and −5 volts. Electrical isolation is achieved with the use of a linear photovoltaic isolation amplifier comprised of U400, U110 and associated circuit elements. Operational amplifiers U400 and U110 are low noise types such as the OP1177, and U401 is an optocoupler designed for use in linear applications such as the IL300 manufactured by Vishay Semiconductor GmbH, Heilbronn, Germany.

In FIG. 19, the signal loop circuits are identical to those described above in respect of FIG. 17 and the same reference numerals have been used to denote like components. However, reference loop circuits of FIG. 19 differ from the reference loop circuits described above in connection with FIG. 17 in that the second terminal of the resistor R202A is not connected directly to circuit ground in cable connector 1100, but is connected to the first terminal of a further capacitor C400C in the amplifier cable and the second terminal of the capacitor C400C is connected to circuit ground. Also, in the circuit of FIG. 19, the second terminal of the resistor R202A is also connected to a first terminal of a capacitor C402C. The second terminal of the capacitor C402C is connected to circuit ground in the shielded filter enclosure.

The first terminal of the capacitor C402C is connected to the first terminal of a capacitor C404C and to the non-inverting input of the amplifier U400A. The second terminal of the capacitor C404C is connected to circuit ground. The non-inverting input of the amplifier U400A is connected to V_{isoground}. The inverting input of the amplifier U400A is connected to the first terminal of a capacitor C406 and the second terminal is connected to the second terminal of the resistor R204B. The second terminal of the capacitor C406 is connected to the output of the amplifier U400A.

The positive power pin of the amplifier U400A is connected to Vᵢₛₒ₊ and the negative power pin of U400A is connected to Vᵢₛₒ₋. The output of the amplifier U400A is connected to the base of a transistor. Q1. The collector of the transistor Q1 is connected to V_{isoground} and to the pin 4 of an amplifier U401A. The emitter of the transistor Q1 is connected to pin 1 of the amplifier U401A. The inverting input of the amplifier U400A is connected to pin 3 of the amplifier U401A and to the first terminal of a resistor R410. The second terminal of the resistor R410 is connected to Vᵢₛₒ₊. Pin 2 of the amplifier U401A is connected to the first terminal of a resistor R412 and the second terminal of R412 is connected to Vᵢₛₒ₊. Pin 5 of the amplifier U401A is connected to circuit ground. Pin 6 of the amplifier U401A is connected to the inverting input of an amplifier unit U110A and to the first terminal of a resistor R413A. The second terminal of the resistor R413A is connected to +5 volts. The non-inverting input of the amplifier U110A is connected to circuit ground. The inverting input of the amplifier U110A is connected to the first terminal of a resistor R213A and to the first terminal of a capacitor C410. The second terminal of the resistor R213A is connected to the first terminal of a resistor R214A and to the second terminal of a capacitor C410 as well as to the output of an amplifier U110A.

As shown in FIG. 19, each reference loop has an isolated ground wire with rf filtering. To obtain maximum isolation, it is preferable to terminate all reference loops on one Isolated ground wire. This is similar to the embodiment described previously consisting of a signal wire and reference wire for each channel, and two ground wires total for all the signal and reference loops. The signal loops ground wire is connected to ground electrode R39 attached to the body, and the reference loops (isolated) ground wire is connected to the reference mesh near the body ground electrode R39 and electrically insulated from the body.

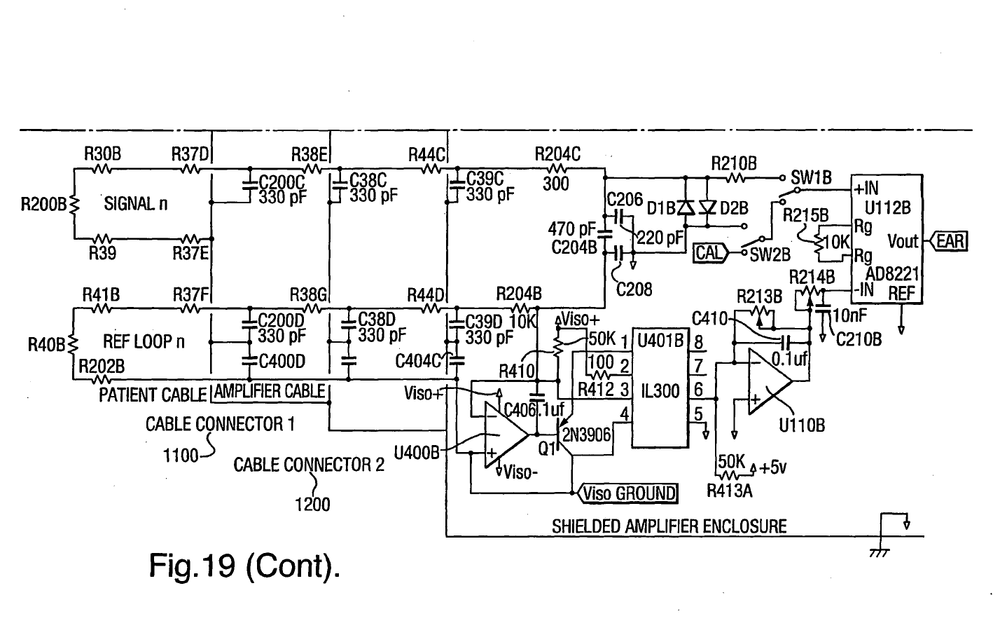

In FIG. 20, an electrode support cap 2010 in accordance with an embodiment of the present invention is shown in place on the head 2030 of a subject. It comprises a flexible head covering piece 2050 provided with holes such as 2070 etc for the ears. The cap is retained on the head by means of a chin strap 2090. Four measurement signal/reference node pairs are provided spatially separated over the surface of the cap, denoted by reference numerals 2110, 2130, 2150 and 2170. Each of these pairs is connected to external circuitry by means of twisted wire pairs 2190, 2210, 2230, 2250.

A separate compensation electrode with associated reference electrode with its own twisted wire pair for external connection is denoted by numeral 2270. This is located just behind the right ear.

At the base of the neck region of the headpiece 2050, is arranged a ground electrode/reference electrode pair 2290, again with a twisted wire pair connection to remote circuitry.

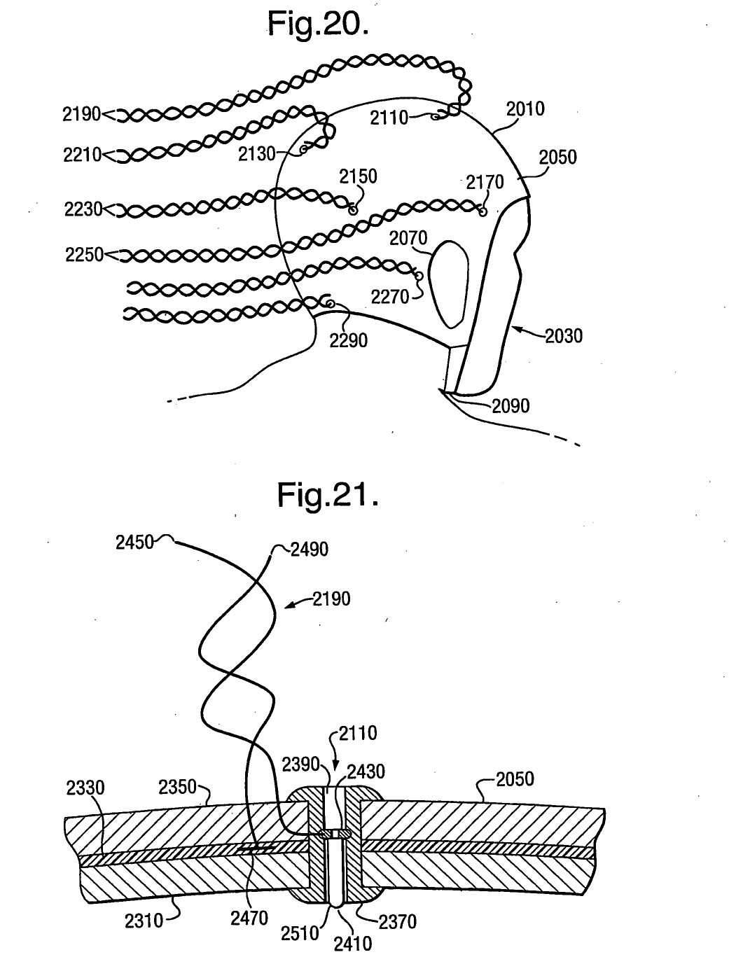

A cross-section through one measurement electrode/reference node pair 2110 is shown in FIG. 21.

As can be seen in this cross-sectional view, the flexible cap headpiece 2050 comprises an insulating nylon stretch fabric base layer 2310, on top of which is situated a silver coated nylon reference mesh 2230. Above this, is situated an upper stretch fabric netting 2350.

This three layer structure 2310, 2330, 2350 is provided with a hole bridged by a cylindrical grommet 2370 of suitable insulating material. A central bore 2390 runs axially through the centre of the grommet. The lower part of this bore is filled with a conductive gel 2410, on top of and in electrical contact therewith, being a measurement electrode metal or carbon insert 2430 which exits the side wall of the grommet, upwardly through the stretch fabric netting layer 2350 to be connected to measurement signal wire 2450 forming one half of the twisted wire pair 2190.

Immediately adjacent the grommet 2370 is located a reference electrode (node) connection 2470, embedded in the conductive silver coated reference mesh layer 2330, which is in electrical contact with wire 2490 which exits through the upper stretch fabric netting 2350, twisted with the measurement signal wire 2450 to form the other half of twisted wire pair 2190.

In use, the lower part 2510 of the conductive gel 2410 is in contact with the scalp of the subject.

In the light of the described embodiments, modifications of those embodiments, as well as other embodiments, all within the scope of the appended claims as interpreted in the light of the specification as a whole and with the knowledge of a person skilled in the art, will now become apparent.

1. An electronic apparatus for reducing interference in a desired signal, the apparatus comprising:—
(a) a plurality of measurement signal lines, each connected to a respective measurement signal electrode; and
(b) one or more reference signal lines, each connected to a respective one or more reference electrodes;
each of said measurement signal lines or a respective group of said measurement signal lines being associated by being in close physical proximity with a respective one of said reference signal lines for a substantial part of their lengths, so that each measurement signal line or signal line group with its corresponding reference signal line forms a measurement signal line or measurement signal line group/reference signal line pair, said electronic apparatus further comprising subtraction means for subtracting an interference signal on each reference signal line from an interference signal on the associated measurement signal line or from each measurement signal line in the measurement signal line group in that measurement signal line or measurement signal line group/reference signal line pair;
wherein at least one of the measurement signal electrodes is arranged to be in direct electrical connection with a subject and at least one of the reference signal electrodes is arranged to be in close physical proximity but not in direct electrical contact with the subject.

2. An electronic apparatus according to claim 1 further comprising an electrically conductive mesh comprising one or more of said reference electrodes.

3. An apparatus according to claim 2 wherein an insulating layer is provided for insulating the conductive mesh from a subject.

4. An apparatus according to any one of claim 2 or 3 wherein said conductive mesh comprises a continuous laminar member.

5. An apparatus according to any one of claim 2 or 3 wherein said conductive mesh comprises a matrix of discrete members respectively comprising said reference electrodes.

6. An apparatus according to any of claims 2 to 5 further comprising an electrode support structure for supporting said electrodes and said conductive mesh.

7. An apparatus according to claim 6, wherein said electrode support structure comprises a flexible cap.

8. An apparatus according to claim 6 wherein said electrode support structure comprises a rigid cap for supporting said electrodes, said conductive mesh being flexible.

9. An apparatus according to any one of claims 6 to 8, wherein said electrode support structure is arranged to effect an EPM.

10. An apparatus according to any one of claims 6 to 9, wherein the electrode support structure apparatus further comprises an electrode support having supported thereon, an array of said measurement signal electrodes presented for contacting the skin of a subject, first connection means being provided for independent electrical connection to each of said measurement signal electrodes, the electrically conductive mesh further having second connection means for independent electrical connection to the or each of said reference electrodes.

11. An apparatus according to any one of claim 9 or 10, wherein the number of said reference nodes is substantially the same as the number of said measurement signal electrodes.

12. An apparatus according to any of claims 9 to 11, wherein each measurement signal electrode or group of signal electrodes has a corresponding respective reference electrode in close physical proximity thereto.

13. An apparatus according to any of claims 9 to 12, wherein said electrode support further supports one or more ground electrodes presented for contacting the skin of a subject, the apparatus further comprising third connection means for independent electrical connection to each of said ground electrode or electrodes.

14. An apparatus according to any of claims 9 to 13, wherein the electrode support supports a single ground electrode.

15. An apparatus according to any of claims 9 to 14, wherein the electrode support supports a compensation signal electrode.

16. An apparatus according to claim 15 when dependent upon claim 14, wherein a respective reference electrode with its own independent electrical connection is provided for the ground electrode and the compensation signal electrode.

17. An apparatus according to any one of the preceding claims, wherein a respective ground line is arranged in associated close proximity with the or each signal line along a substantial part of the length thereof, each of the ground lines being connected to one or more ground electrodes in direct or indirect electrical contact with the subject.

18. An electronic apparatus according to claim 17, further comprising a further ground line arranged in associated close proximity with the or each reference signal line along a substantial part of the length thereof.

19. An electronic apparatus according to any one of the preceding claims, wherein the interference comprises a plurality of interference components, the apparatus further comprising an electronic circuit which comprises:
(a) at least one primary signal processing unit, the or each primary signal processing unit having a respective measurement signal input for receiving a respective one of said measurement signal or signals and the or each primary signal processing unit comprising a plurality of interference reduction modules; and
(b) a respective compensation signal component input for each interference reduction module.

20. An electronic apparatus according to claim 19, wherein the compensation signal input is connected via a compensation signal line to a compensation signal electrode in direct electrical connection with a subject and a circuit ground connection is connected via a ground line to a ground electrode, respective reference signal lines being arranged in close proximity with the compensation signal line and ground line along respective substantial parts of the length thereof, the reference signal lines being connected to further respective reference electrodes.

21. An electronic apparatus according to any one of claim 19 or 20, further comprising:
(a) a compensation signal processing unit having a compensation signal input and comprising means for deriving from a compensation signal, a plurality of compensation signal components each of which is related to a respective one or more of the interference components; and
(b) the compensation signal processing unit also having a respective compensation signal component output for each compensation signal component, each said output being respectively connected to one of the compensation signal component inputs.

22. An electronic apparatus according to claim 21, wherein in each primary signal processing unit, the interference reduction modules are arranged in series.

23. An electronic apparatus according to claim 21 or claim 22, wherein in each primary signal processing unit, respective interference reduction modules are provided for reduction of at least two of rf interference, magnetic field switching interference, mains power interference, eyeblink artifact interference and ballistocardiogram interference, respectively.

24. An electronic apparatus according to any one of claims 21 to 23, wherein a respective measurement signal electrode is connected to the or each measurement signal input of the at least one primary signal processing unit via a measurement signal line and is in direct electrical contact with a subject and for each measurement signal line or group of signal lines, a corresponding reference signal electrode is connected via a reference signal line to a respective reference signal input of the at least one primary signal processing unit.

25. An electronic apparatus according to claim 24, wherein the or each primary signal unit further comprises subtraction means for subtracting at least part of a signal on the respective reference signal line from the signal on the corresponding respective measurement signal line or lines.

26. An electronic apparatus according to claim 24, wherein the or each primary signal unit further comprises subtraction means for subtracting at least part of one or more of the compensation signal components from the signal on the corresponding respective measurement signal line or lines.

27. An electronic apparatus according to any one of claims 21 to 26, wherein said compensation signal processing unit has a separate circuit ground connection.

28. An electronic apparatus according to claim 24 or claim 25, wherein a respective signal ground line is associated in close proximity with the or each measurement signal line/reference line pair along a substantial part of the length thereof, each of the ground lines being connected to one or more ground electrodes in direct or indirect electrical contact with the subject.

29. An electronic apparatus according to claim 28, wherein the circuit ground connections of the ground lines associated with the signal lines and associated grounds are electrically isolated from the circuit ground connections of the reference lines.

30. An electronic apparatus according to claim 20, wherein each measurement signal line is twisted together with its respective reference line and the ground signal line and compensation signal line are twisted together with their respective reference lines.

31. An electronic apparatus according to claim 30 where all of the measurement signal line/reference line pairs, the compensation signal line reference line pair and the ground line/reference line pair are twisted together.

32. An electronic apparatus according to claim 17, wherein each measurement signal line and associated ground line are respectively twisted together and each reference line and associated ground line are respectively twisted together.

33. An electronic apparatus according to claim 32, wherein each measurement signal line/ground line twisted pair and each associated compensation signal line/ground line twisted pair are respectively twisted together.

34. An electronic apparatus according to claim 29, wherein each associated measurement signal line, reference signal line and ground line are twisted together.

35. An electronic apparatus according to any one of claims 17, 20, or 28 to 34, wherein the or each measurement signal line/reference signal line pair is shielded.

36. An electronic apparatus according to any of claims 17, 20, 24, 25 or 28 to 35, wherein for at least some signal line/reference line pairs, at least one additional reference line is provided, connected to the same or a respective further reference electrode.

37. A combined measurement apparatus comprising an MRI or TMS unit and an EPM system which comprises an electronic apparatus for reducing interference according to any preceding claim.

38. A combined apparatus according to claim 37, wherein the MRI unit is adapted for fMRI.

39. A combined apparatus according to claim 37 or claim 38, wherein the EPM system is selected from systems for effecting one or more of EEG, ECG, EMG, EOG, ERG and GSR.

40. A method of reducing interference from a desired signal, the method comprising
(a) providing a plurality of measurement signal lines, each carrying a desired signal and an interference signal;
(b) providing one or more reference signal lines, each carrying at least an interference signal, each measurement signal line or a respective group of measurement signal lines being associated by being in close physical proximity for a substantial part of its length with a respective reference signal line to provide respective measurement signal line or measurement signal line group/reference signal line pairs; and
(c) performing a subtraction step of subtracting the interference signal on each respective reference signal line from the interference signal on the associated measurement signal line or from each measurement signal line in the measurement signal line group of its measurement signal line or measurement signal line group/reference line pair;
wherein at least one of the measurement signal electrodes is arranged to be in direct electrical connection with a subject and at least one of the reference signal electrodes is arranged to be in close physical proximity but not in direct electrical contact with the subject.

41. A method of reducing interference from a desired signal, the method comprising
(a) providing a signal line carrying a desired signal and an interference signal;
(b) providing a reference line carrying at least an interference signal, said signal line and reference line being associated by being in close physical proximity for a substantial part of their lengths; and
(c) a subtraction step of subtracting the interference signal on the reference line from the interference signal on the signal line.

42. A method according to claim 40 or 41, further comprising:
(a) deriving a compensation signal; and
(b) generating a plurality of compensation signal components from said compensation signal;
wherein the subtraction step comprises separately subtracting at least part of each of said compensation signal components from said measurement signal.

43. An electronic apparatus for reducing interference in a desired signal, the apparatus comprising
(a) a signal line connected to a signal electrode; and
(b) a reference line connected to a reference electrode;
said signal line and reference line being associated by being in close physical proximity for a substantial part of their lengths, said electronic apparatus further comprising subtraction means for subtracting an interference signal on the reference line from an interference signal on the signal line thereby to enhance a desired signal on the signal line.

44. An electronic apparatus for reducing interference in a signal derived from an EPM the apparatus comprising
(a) a signal line connected to a signal electrode;
(b) a reference line connected to a reference electrode; and
(c) at least one ground line for said signal line and reference line, said ground line or lines being connected to at least one ground electrode or individually to respective ground electrodes;
said electronic apparatus further comprising subtraction means for subtracting an interference signal on the reference line from a signal on the signal line.

45. An electronic apparatus for reducing interference in a desired signal, the apparatus comprising:—
(a) a plurality of signal lines, each connected to a respective signal electrode; and
(b) one or more reference lines connected to one or more reference electrodes; and;
(c) one or more ground lines connected to one or more ground electrodes;
said electronic apparatus further comprising subtraction means for subtracting an interference signal on the or each reference line from an interference signal on the signal lines and/or subtracting an interference signal on the or each ground line from the interference signal on the signal lines.

46. A method of reducing interference on a signal derived from an EPM, the method comprising
(a) providing a signal line carrying a desired signal and a first interference signal, said signal line being connected to a signal electrode;
(b) providing a reference line carrying at least a second interference signal, said reference line being connected to a reference electrode;
(c) providing a ground line for said signal line and reference line, said ground line or lines being connected to at least one ground electrode or individually to respective ground electrodes; and
(d) a subtraction step of subtracting the second interference signal on the reference line from the first interference signal on the signal line.

47. A method of reducing interference from a desired signal, the method comprising
(a) providing a plurality of signal lines, each carrying a desired signal and a first interference signal;
(b) providing one or more reference lines carrying at least a second interference signal;
(c) providing one or more ground lines; and
(d) performing a subtraction step of subtracting the second interference signal from said first interference signal.

48. An electronic apparatus for reducing interference in a desired signal, the apparatus comprising:—
(a) a plurality of measurement signal lines, each connected to a respective measurement signal electrode; and
(b) one or more reference signal lines, each connected to a respective one or more reference electrodes;
each of said measurement signal lines being associated by being in close physical proximity with a respective one or more of said reference signal lines for a substantial part of their lengths, so that each measurement signal line with its corresponding reference signal line forms a measurement signal line/reference signal line pair, said electronic apparatus further comprising subtraction means for subtracting an interference signal on each reference signal line or lines from an Interference signal on the associated measurement signal line in that measurement signal line/reference signal line pair;
wherein at least one of the measurement signal electrodes is arranged to be in direct electrical connection with a subject and at least one of the reference signal electrodes is arranged to be in close physical proximity but not in direct electrical contact with the subject.

49. A cap for supporting one or more electrodes for use in an electronic apparatus for reducing interference in a desired signal, the cap comprising:—
(a) a conductive layer; and
(b) at least one measurement signal electrode positioned for contact with a subject; at least one of the at least one measurement signal electrode or electrodes having associated therewith a reference electrode in electrical contact with the conductive layer but arranged so as not to be in use in direct electrical contact with the subject.

50. A cap according to claim 49, wherein the conductive layer comprises a conductive mesh.

51. A cap according to any one of claim 49 or 50, wherein the cap comprises an electrode support structure apparatus for effecting an EPM, the cap further comprising:
an array of measurement signal electrodes presented for contacting the skin of a subject, first connection means being provided for independent electrical connection to each of said measurement signal electrodes, and
second connection means for independent electrical connection to the or each of said reference electrodes.

52. A cap according to any one of claims 49 to 51, wherein an insulating layer is provided for insulating in use the conductive layer from the subject.

53. A cap according to any one of claims 49 to 52, wherein the number of said reference electrodes is substantially the same as the number of said measurement signal electrodes.

54. A cap according to any one of claims 49 to 53, wherein each measurement signal electrode or group of signal electrodes has a corresponding respective reference electrode in close physical proximity thereto.

55. A cap according to any one of claims 49 to 54, wherein said cap further supports one or more ground electrodes presented for contacting the skin of the subject in use, the cap further comprising third connection means for independent electrical connection to each of said ground electrode or electrodes.

56. A cap according to any one of claims 49 to 55, wherein the cap supports a single ground electrode.

57. A cap according to any of claims 49 to 56, wherein the cap supports a compensation signal electrode.

58. A cap according to claim 57 when dependent upon claim 56, wherein a respective reference electrode with its own independent electrical connection is provided for the ground electrode and the compensation signal electrode.

59. A cap according to any one of claims 49 to 58, wherein said conductive layer comprises a continuous laminar member comprising one or more of said reference electrode or electrodes.

60. A cap according to any of claims 49 to 58, wherein said conductive layer comprises a matrix of discrete members respectively comprising one or more of said reference electrode or electrodes.

61. A cap according to any of claims 49 to 60, wherein said cap is a flexible cap.

62. A cap according to any of claims 49 to 60, wherein said cap is a rigid cap, the conductive layer being flexible.
